(19) United States
(12) Patent Application Publication
(10) Pub. No.: US 2026/0290623 A1
(43) Pub. Date: Sep. 24, 2026

(54) SUGGESTING REFERENCE CONTENT ITEMS FOR A MEDICAL PRACTITIONER THROUGH AN ONLINE COLLABORATIVE MEDICAL PLATFORM

(71) Applicant: Cilag GmbH International, Zug (CH)

(72) Inventors: Shan Gowri Jegatheeswaran, Santa Clara, CA (US); Raymond Samuel Fryrear, II, Cincinnati, OH (US); Philippa Erin Kay Gray, San Diego, CA (US); Fiona Middlemiss Haig, York (GB); Tyler Lee Hartley, Seattle, WA (US); Alexandre Hennen, New York, NY (US); Vikram Mohan, San Francisco, CA (US); Asad Raza, Lewisville, TX (US); Jamie Gavin Roots, Madrid (ES)

(21) Appl. No.: 19/689,844

(22) Filed: May 27, 2026

Related U.S. Application Data
(63) Continuation of application No. PCT/IB2024/062180, filed on Dec. 4, 2024.
(60) Provisional application No. 63/605,879, filed on Dec. 4, 2023.
(60) Provisional application No. 63/641,754, filed on May 2, 2024.
(60) Provisional application No. 63/661,015, filed on Jun. 17, 2024.
(60) Provisional application No. 63/661,858, filed on Jun. 19, 2024.
(60) Provisional application No. 63/717,950, filed on Nov. 8, 2024.
(60) Provisional application No. 63/718,000, filed on Nov. 8, 2024.
(60) Provisional application No. 63/719,015, filed on Nov. 11, 2024.

(51) Int. Cl. G16H 80/00

(52) U.S. Cl. CPC G16H 80/00

(57) ABSTRACT

A collaborative medical platform facilitates remote collaboration relating to medical procedures during preprocedural, intraprocedural, and postprocedural stages of a medical case. The collaborative medical platform selects and presents reference content items, such as video and associated data of a previously completed medical procedure, relevant to one or more medical procedures to a medical practitioner. Based on characteristics of a medical practitioner, the collaborative medical platform selects one or more reference content items with attributes matching, or otherwise related to, at least one characteristic of the medical practitioner. Prior interactions by the medical practitioner with reference content items may be leveraged to select reference content items or to determine when to present a selected reference content item to the medical practitioner.

### CROSS-REFERENCE TO RELATED APPLICATIONS

This application is a continuation of PCT Application No. PCT/IB2024/062180 filed on Dec. 4, 2024, which claims the benefit of the following applications: U.S. Provisional Patent Application No. 63/605,879 filed on Dec. 4, 2023, U.S. Provisional Patent Application No. 63/641,754 filed on May 2, 2024, U.S. Provisional Patent Application No. 63/661,015 filed on Jun. 17, 2024, U.S. Provisional Patent Application No. 63/661,858 filed on Jun. 19, 2024, U.S. Provisional Patent Application No. 63/717,950 filed on Nov. 8, 2024, U.S. Provisional Patent Application No. 63/718,000 filed on Nov. 8, 2024, and U.S. Provisional Patent Application No. 63/719,015 filed on Nov. 11, 2024. The contents of each of the foregoing applications are incorporated by reference herein.

### BACKGROUND

### Technical Field

The described embodiments relate to a system and method for selecting and providing reference content about one or more medical procedures to a medical practitioner through a collaborative medical platform that facilitates collaboration between medical practitioners.

### DESCRIPTION of the RELATED ART

When preparing for various medical procedures, reviewing reference content may increase efficiency of procedures and may improve patient results. However, reference content may be provided in various formats—text articles, video presentations, audio presentations—and stored in disparate locations. This increases an amount of time for a medical practitioner to identify and to access reference content most relevant to certain medical procedures.

### BRIEF DESCRIPTION OF THE DRAWINGS

FIG. 1 is an example embodiment of a computing environment for an electronically-assisted medical procedure.

FIG. 2 is a block diagram of an example architecture for a collaborative medical platform.

FIG. 3A shows a first view of an example practitioner dashboard associated with a collaborative medical platform.

FIG. 3B shows a second view of an example practitioner dashboard associated with a collaborative medical platform.

FIG. 4 shows an example practitioner dashboard displaying a suggested reference content item to a practitioner associated with a collaborative medical platform.

FIG. 5 is an example embodiment of a case sharing interface associated with sharing a medical case in the collaborative medical platform.

FIG. 6 is an example embodiment of a case dashboard associated with a set of cases in a collaborative medical platform.

FIG. 7 is an example telepresence interface associated with a collaborative medical platform.

FIG. 8 is another example of a telepresence interface associated with a collaborative medical platform.

FIG. 9 is an example analytics dashboard associated with a collaborative medical platform.

FIG. 10 is an example video interface associated with a collaborative medical platform.

FIG. 11 is a flowchart of an example embodiment of a process for selecting a reference content item for presentation to a medical practitioner via a collaborative medical platform.

### DETAILED DESCRIPTION

The (FIGS.) and the following description describe certain embodiments by way of illustration only. One skilled in the art will readily recognize from the following description that alternative embodiments of the structures and methods illustrated herein may be employed without departing from the principles described herein. Reference will now be made to several embodiments, examples of which are illustrated in the accompanying figures. Wherever practicable, similar or like reference numbers may be used in the figures and may indicate similar or like functionality.

A collaborative medical platform facilitates exchange of data between remote medical practitioners in relation to medical cases during preprocedural, intraprocedural, and postprocedural stages. The collaborative medical platform stores or enables access to patient records, imaging data, video data, telemetry data from medical equipment, biometric sensor data from patients, and other medical information that may be obtained prior to medical procedures being performed, during medical procedures, or after medical procedures are performed. The collaborative medical platform includes various interfaces that enable collaboration between medical practitioners via shared media libraries, content feeds associated with specified cases, telepresence sessions recorded during procedures or simulations of procedures, and various analytics associated with procedures. The collaborative medical platform may furthermore intelligently select one or more reference content items for a medical practitioner. A reference content item comprises video data, audio data, text data, or a combination thereof including information about a medical procedure. For example, a reference content item is a video recording of a previously performed medical procedure with patient identifying information removed. The collaborative medical platform leverages information about the medical practitioner, such as areas of specialization, medical procedures previously performed by the medical practitioner, connections between the medical practitioner and other medical practitioners, types of reference content items previously accessed by the medical practitioner, or other information about the medical practitioner. Further, the collaborative medical platform may dynamically identify one or more reference content items to the medical practitioner to simplify retrieval of the one or more reference content items.

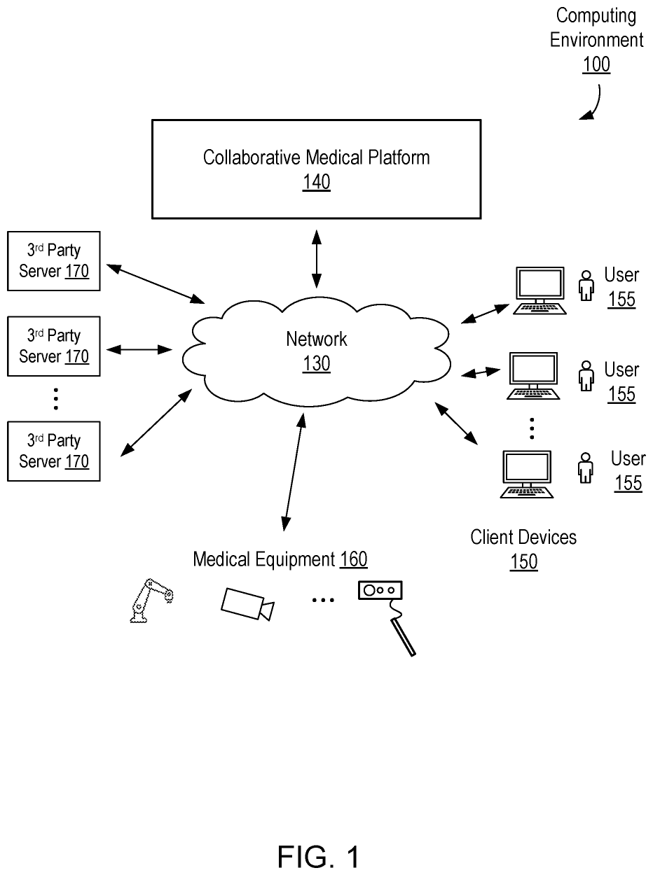

FIG. 1 illustrates an example embodiment of a computing environment 100 for a collaborative medical platform 140. The collaborative medical platform 140 may include one or more servers that are coupled by a network 130 to client devices 150 associated with users 155 of the collaborative medical platform 140, medical equipment 160, and various third-party servers 170. The collaborative medical platform 140 facilitates collaborative exchange of data between medical practitioners, patients, administrators, or other users 155 via the client devices 150 in support of preprocedural, intraprocedural, and postprocedural stages of medical cases. The collaborative medical platform 140 may furthermore facilitate access to telemetry data from medical equipment 160 (including, for example, real-time video, images, biometric sensing data, equipment control and/or status signals, etc.) that may be utilized in conjunction with performing medical procedures and managing patient cases. Furthermore, the collaborative medical platform 140 may facilitate access to various third-party servers 170 that provide external services such as, for example, electronic healthcare records (EHR) services, medical telepresence services, operating room scheduling, data analytics services, etc.

In support of a preprocedural stage of a medical case, the collaborative medical platform 140 provides an online platform that enables medical practitioners to prepare for performance of the medical procedure. Here, the collaborative medical platform 140 may provide access to various text and multimedia resources relating to the planned procedure such as video libraries, instructional information, statistical information, relevant research information, or other resources. The collaborative medical platform 140 may furthermore enable a case owner to selectively share the case with one or more remote medical practitioners to facilitate collaboration. Here, the collaborative medical platform 140 provides interfaces for facilitating interactions between remote practitioners through a case-specific content feed and enabling sharing of case information, commentary, multimedia, or other information. In an embodiment, the collaborative medical platform may automatically redact patient identifiers or selective patient information to maintain data privacy. The collaborative medical platform 140 tracks all information in association with a case identifier to maintain a complete record of case data, multimedia, collaborative content feeds, or other information collected by the collaborative medical platform 140.

To further support the preprocedural stage of a medical case, the collaborative medical platform 140 may select one or more reference content items for presentation to a medical practitioner before performing a medical procedure. In various embodiments, the collaborative medical platform 140 maintains a store or a library of reference content items from which reference content items for the medical practitioner are selected. Alternatively or additionally, one or more third-party servers 170 maintain reference content items, and the collaborative medical platform 140 selects one or more reference content items from a third-party server 170. Reference content items for a medical practitioner may be retrieved from a combination of one or more third-party servers 170 and the collaborative medical platform 140. The collaborative medical platform 140 leverages data in a user profile of a medical practitioner to select one or more reference content items for the medical practitioner.

In support of an intraprocedural stage of a medical case, the collaborative medical platform 140 may facilitate presentation of various information to support the procedure such as preprocedural images, models, patient data, equipment information, or other data. The collaborative medical platform 140 may furthermore facilitate a telepresence session that enables one or more remote contributors to access video, images, 3D models, equipment telemetry data, or other data streams capturing during an ongoing medical procedure. The collaborative medical platform 140 may furthermore enable remote practitioners to provide annotations or other commentary related to real-time video, images, or three-dimensional models associated with a procedure. The collaborative medical platform 140 tracks and stores all data from the procedure (including video, medical equipment telemetry, and collaborative commentary) in association with the case identifier to enable subsequent access.

In support of a post-operative stage of a medical procedure, the collaborative medical platform 140 enables medical practitioners connected with a case to collaboratively monitor data associated with a patient's recovery. For example, the collaborative medical platform 140 may provide interfaces for viewing health records associated with the patient's recovery and facilitate collaborative exchange between medical practitioners through a case-specific content feed. The collaborative medical platform 140 may furthermore perform various analytics relating to performed medical procedures based on aggregations of data. The analytics may be useful to support patient recovery, to improve future procedures, and to track the performance of medical practitioners.

The collaborative medical platform 140 may intelligently utilize data collected during preprocedural, intraprocedural, and/or postprocedural stages of a case during a different stage of the same case or other cases. For example, annotations of images or 3D models, practitioner comments from a content feed, or other information obtained during a preprocedural stage may be made available in the intraprocedural stage to aid the performing practitioner through the procedure. Analytical data relating to postprocedural data may be utilized to generate recommendations for future procedures in order to improve efficiencies and/or outcomes.

The collaborative medical platform 140 may also facilitate functions such as managing clinical trials, facilitating education training and performance tracking, facilitating broadcasts of medical-related presentations, and facilitating procedure scheduling. Beneficially, the collaborative medical platform 140 stores complete records of medical cases (including video and telemetry from procedures) in a centralized and standardized platform that naturally allows for collaboration in an online environment, where practitioners may interact from disparate remote locations. The collaborative medical platform 140 may maintain data in a manner that adheres to data privacy and compliance obligations of medical practitioners and organizations.

The collaborative medical platform 140 may furthermore employ various machine learning techniques to infer recommendations, insights, or other artificially generated contributions based on the data collected into the collaborative medical platform 140. For example, the collaborative medical platform 140 may generate a recommendation for a medical practitioner to review a reference content item relevant to a medical practitioner based on stored information for the medical practitioner. For example, the collaborative medical platform 140 generates a recommendation for the medical practitioner based on a type of procedure scheduled to be performed by the medical practitioner; in various embodiments, the recommended content item comprises case records associated with one or more historical cases captured in the collaborative medical platform 140 relating to prior performances of the type of procedure on a similarly situated patient. If granted appropriate permissions, the practitioner may then review an entire case record through the collaborative medical platform 140 including preprocedural information, videos or other data from the procedure itself, and postprocedural outcome data. In another example, the collaborative medical platform 140 may intelligently generate a recommendation to invite a particular medical practitioner to collaborate on a case based on that practitioner having relevant expertise, experience, and/or availability. An invitation may then be generated to the medical practitioner to enable access and collaboration on the case during at least one of the preprocedural, intraprocedural, and postprocedural stages. Furthermore, the collaborative medical platform 140 may intelligently identify and present patient risk factors relevant to procedure performance, planning, and postprocedural care. The collaborative medical platform 140 may also intelligently recommend training for medical practitioners based on their individual tracked performance and various comparative analytics.

The collaborative medical platform 140 may be implemented using on-site computing or storage systems, cloud computing or storage systems, or a combination thereof and may be implemented utilizing local or cloud-based servers, which may include physical or virtual machines, or a combination thereof. Cloud-based servers may include private cloud systems, public cloud systems, hybrid public/private cloud systems, or a combination thereof. Accordingly, the collaborative medical platform 140 may be local, remote, and/or distributed relative to the medical environments where procedures are performed and relative to the client devices 150 providing user access. Furthermore, different portions of the collaborative medical platform 140 may execute on different remote servers and various system elements of the collaborative medical platform 140 may be communicatively coupled over a network 130.

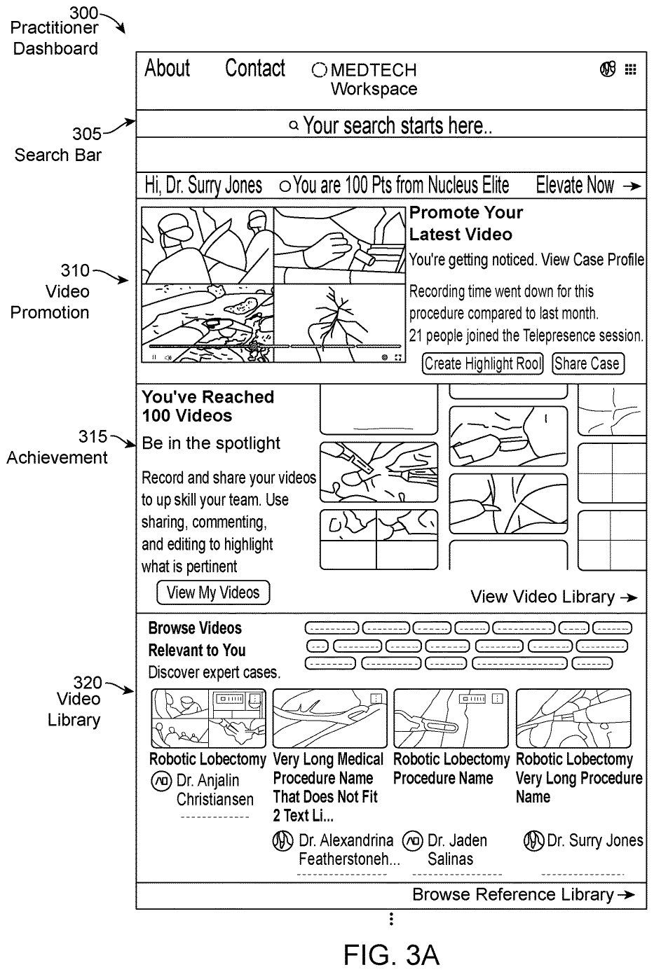

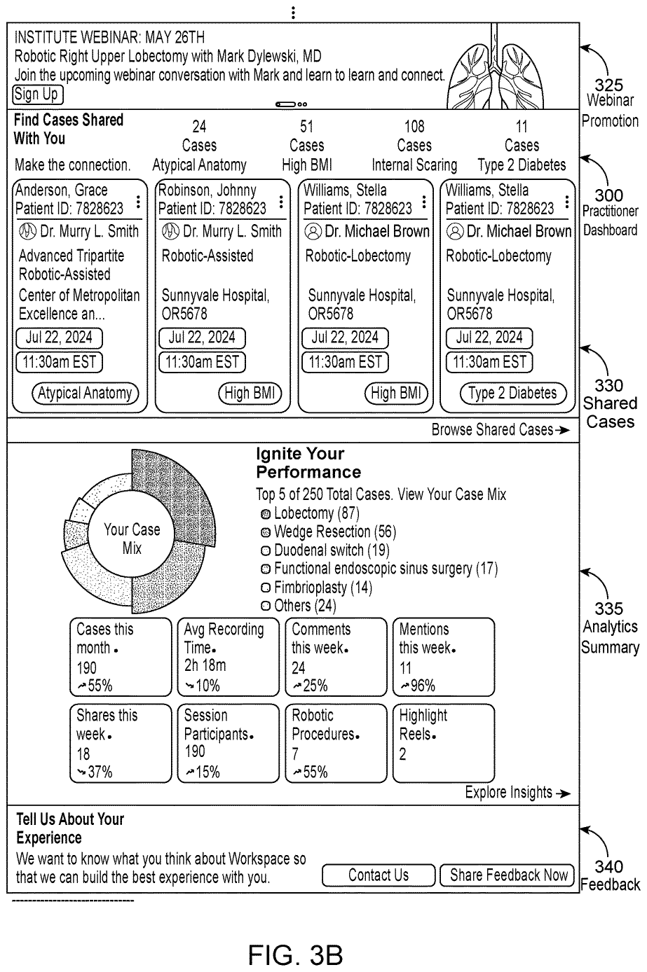

The client devices 150 may include, for example, a mobile phone, a tablet, a laptop or desktop computer, other computing device, or application executing thereon for accessing the collaborative medical platform 140 via the network 130. The client devices 150 may enable access to various user interfaces (which may comprise web-based interfaces accessed via a browser or application interfaces accessed via an application) for viewing and/or editing information associated with the collaborative medical platform 140. The client devices 150 may include conventional computer hardware such as a display, input device (e.g., touch screen), memory, a processor, and a non-transitory computer-readable storage medium that stores instructions for execution by the processor in order to carry out functions described herein. Examples of user interfaces are described in further detail below with respect to FIGS. 3-10.

The third-party servers 170 may facilitate diverse services utilized by the collaborative medical platform 140. For example, the third-party servers 170 may include various EHR systems for managing patient records, robotic control platforms for controlling surgical robots or other medical equipment, telepresence servers for facilitating telepresence services, patient scheduling systems, hospital information systems (HIS), or other servers. As another example, one or more third-party servers 170 include reference content items about various medical procedures, such as articles about various medical procedures, audio data related to medical procedures, video data related to medical procedures, or other descriptive information about medical procedures. The third-party servers 170 may be implemented using various on-site computing or storage systems, cloud computing or storage systems such as private cloud systems, public cloud systems, hybrid public/private cloud systems, or a combination thereof.

The medical equipment 160 may include various sensors such as cameras or other imaging equipment, biometric monitors, or other sensing devices that collect data associated with a medical procedure being performed. Sensor data may include physiological or biological signals (such as pulse rate, blood pressure, body temperature, etc.), video, electrical signals representative of a state of a medical instruction, or other information. Cameras or image sensors may include still image cameras, video cameras, 3-dimensional (3D) imaging devices, or a combination thereof. The cameras can include stationary cameras in a medical environment (e.g., operating room) or may include cameras integrated into medical instruments such as endoscopic cameras. Imaging systems may include computed tomography (CT) imaging systems, medical resonance imaging (MRI) systems, X-ray systems, or other imaging equipment. The medical equipment may furthermore include a robotic device that facilitates robotically-assisted medical procedures. The robotic device may include, for example, a robotic arm or other computer-controlled mechanical device that performs or assists with a medical procedure. The robotic device may be pre-programmed to perform a certain set of steps or tasks, and/or may be manually controlled by an operator. Telemetry data associated with a robotic device may include force data, positional data, or other sensor data, control signals, fault conditions, or other data relating to operation of the robotic device during a procedure. The medical equipment data may be streamed to the collaborative medical platform 140 in real-time or may be stored on a third-party server 170 and later uploaded to the collaborative medical platform 140.

The network 130 comprises communication pathways for communication between the collaborative medical platform 140, the medical equipment 160, the client devices 150, and the third-party servers 170. The network 130 may include one or more local area networks and/or one or more wide area networks (including the Internet). The network 130 may also include one or more direct wired or wireless connections (e.g., Ethernet, WiFi, cellular protocols, WiFi direct, Bluetooth, Universal Serial Bus (USB), or other communication link).

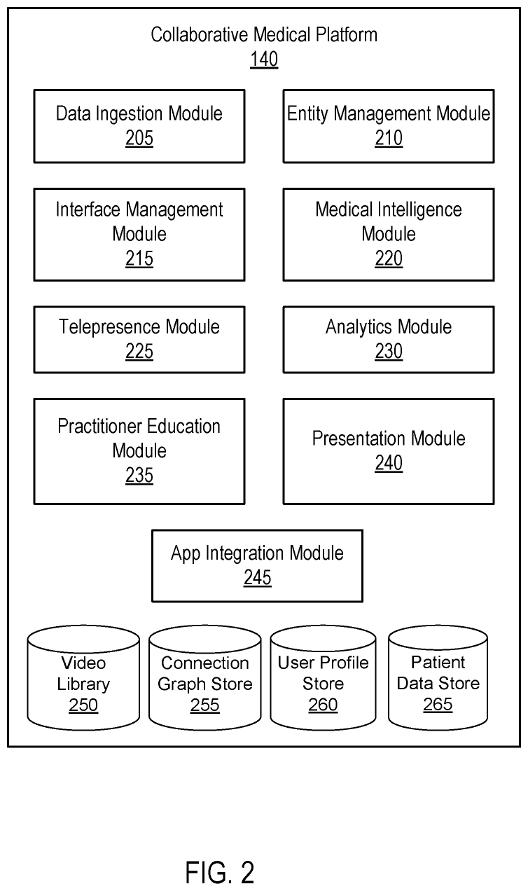

FIG. 2 is a block diagram showing an example architecture of an embodiment of the collaborative medical platform 140. In the embodiment of FIG. 2, the collaborative medical platform 140 includes a data ingestion module 205, an entity management module 210, an interface management module 215, a medical intelligence module 220, a telepresence module 225, an analytics module 230, a practitioner education module 235, a presentation module 240, an application integration module 245, a video library 250, a connection graph store 255, a user profile store 260, and a patient data store 265. In other embodiments, the collaborative medical platform 140 includes different or additional functional blocks than those shown in FIG. 2. Further, in some embodiments, a single functional block provides the functionality of multiple functional blocks shown in FIG. 2.

While in one embodiment, the illustrated functional blocks may execute entirely within the collaborative medical platform 140, alternative embodiments may include various modules or discrete functions of modules being executed by one or more third-party servers 170. Here, the collaborative medical platform 140 may interact with a third-party server 170 via an application programming interface (API) to enable the collaborative medical platform 140 to request and utilize services provided by the third-party servers 170 to facilitate any of the functions described herein. For example, in an embodiment, electronic health records may be provided by a third-party server 170. Here, the collaborative medical platform 140 may query the third-party server 170 for relevant data but does not necessarily locally store complete patient records. Furthermore, third-party servers 170 may facilitate services such as telepresence sessions, presentation creation, access to video resources, three-dimensional model generation, or other aspects of the functions of the collaborative medical platform 140 described herein.

The data ingestion module 205 ingests various medical data used by the collaborative medical platform 140. The data ingestion module 205 may be electronically coupled to one or more external servers, databases, or other data sources that supply the medical data. The medical data may include, for example, profile data for patients (e.g., demographic information, health history, etc.), medical professionals (e.g., expertise, experience, etc.), or facilities, information about medical conditions, procedures, and medications, information about robotic systems, imaging systems, intervention tools, or other medical equipment, information about post-procedural outcomes, or other medical information discussed herein.

The data ingestion module 205 may aggregate data from various input data sources. For example, the data ingestion module 205 may obtain medical data from conventional electronic health records (EHR) systems. Here, the data ingestion module 205 may perform various pre-processing to normalize data to a standardized format used by the collaborative medical platform 140. For example, medical records may be organized in a database structure that includes values (strings, numerical values, binary values, or other data types) assigned to each of a set of predefined information fields.

The data ingestion module 205 may furthermore interface with one or more imaging systems to ingest preprocedural, intraprocedural, or postprocedural images, video, or three-dimensional models associated with patients. For example, the data ingestion module 205 may obtain and store X-ray images, magnetic resonance imaging (MRI) images, computed tomography (CT) scan images, visible light images, near infrared fluorescent (NIRF) images, or other medical images, video, or three-dimensional models derived from them. Image data may furthermore include image or video data from one or more cameras present in a medical environment where a medical procedure is being performed, such as one or more overhead cameras and/or one or more endoscopic cameras. Imaging data may include associated metadata such as telemetry data from one or more medical instruments used to perform the medical procedure, annotations or commentary associated with the video received from one or more medical practitioners associated with the medical procedure, segmentation data associated with dividing a video into segments relating to different steps of a procedure, or other information relating to image or video data.

To simplify subsequent retrieval and review of video of a medical procedure along with associated metadata, the data ingestion module 205 may perform various preprocessing and indexing of the content and associated metadata. For example, the data ingestion module 205 indexes video of a medical procedure with associated metadata to correlate different metadata with different portions of the video, synchronize videos associated with the same medical procedure, or perform various encoding or reformatting of video data. Videos may furthermore be automatically segmented and indexed into video segments corresponding to different steps of a procedure.

The data ingestion module 205 may furthermore integrate with various robotic platforms or other medical equipment to obtain telemetry data associated with procedures. For example, the data ingestion module 205 may obtain various sensor data from sensors utilized during medical procedures, identifying information associated with medical equipment, control data associated with control a robotic platform or other medical equipment, or other data generated from medical equipment in associated with performed medical procedures.

The data ingestion module 205 may furthermore provide interfaces accessible via the client device 150 for ingesting data input directly into the collaborative medical platform 140. For example, the data ingestion module 205 may present various forms or freeform entry elements to enable entry of medical information relevant to operation.

In an embodiment, the data ingestion module 205 may manage data in a manner consistent with various compliance and privacy policies. For example, the data ingestion module 205 may enable removal or redaction of portions of received data to preserve privacy of a patient when the data is used for purposes in which patient identification is not necessary.

The entity management module 210 manages presentation of entity pages associated with different entities affiliated with the collaborative medical platform 140 and manages connections between entities. Entities may include, for example, users 155 (which may medical practitioners, patients, administrators, etc.), medical cases associated with procedures, facilities, medical equipment 160, files or media content, events (e.g., conferences), presentations, training modules, or other data objects. Entity pages may comprise web pages accessible via a web browser of the client device 150 or may comprise pages of a desktop or mobile application installed on a client device 150.

Each entity page for an entity may enable viewing of information associated with the entity and/or interactions with the entity. For example, each user 155 of the collaborative medical platform may have a dedicated page that provides information about the user 155 such as identifying information, role (e.g., surgeon, nurse, executive, administrator, patient, etc.) profile information (e.g., biography, credentials, etc.), assigned cases, procedure histories, connections to other users or cases, scheduling information, or other user-specific data. An entity page for a patient (whether or not the patient is a user 155 of the collaborative medical platform 140) may include patient profile information, health history, planned procedures, risk factors, or the medical information associated with the patient. An entity page for a medical case may include information about a patient associated with the case, descriptive information about a medical procedure (such as a type of medical procedure) associated with the case, a medical environment where the medical procedure is to be performed, other descriptive information about the medical procedure, a status of the procedure (e.g., preprocedural stage, intraprocedural stage, or postprocedural stage), or other information relevant to a medical case. Pages may furthermore include various interactive elements (e.g., content feeds) that enable users to share and interact with data associated with that entity as will be further described below.

The entity management module 210 also organizes pages and associated data received into the collaborative medical platform 140 into a connection graph (stored to the connection graph store 255) that captures relationships between different entities and associated data. Some connections may be configured as default connections, while other connections may be created based on specific actions from users 155. For example, users 155 may be connected by default to other users 155 (with at least viewing permissions) within the same organization. Alternatively, connections may be generated only when a user 155 expressly invites another user 155 to connect and the receiving user 155 accepts the connection request. Connections between medical practitioners and medical cases may similarly be created by default or in response to invitations to create a connection. For example, a default connection may be created between an entry for a planned medical procedure and a medical practitioner assigned responsibility for the procedure. Alternatively, all medical practitioners within an organization or within a relevant department may become connected to a planned procedure as a default. In other scenarios, a user may share a medical case with one or more other medical practitioners to generate a connection request that invites the other medical practitioners to collaborate with on the medical case. Accepting the connection request may then create a connection between the invited practitioner and the medical case. Supplemental connections may also automatically be generated (e.g., between the owner of the procedure and the invited contributor). Connections may furthermore be created between users 155 and individual videos, files, presentations, or other data objects. For example, a user 155 that creates or owns a video may share the video with one or more other users 155 to grant access rights to the video.

Connections between entities may be of diverse types and may be governed by different permissions. Generally, pages may be accessed only by users having appropriate access permissions. Different permission levels may dictate distinct levels of access for different pages. For example, depending on user-specific permissions for a particular page, the user may be permitted or blocked from accessing the data, editing the data, commenting or annotating the data, deleting the data, or performing other modifications. In an embodiment, a page may have a page owner with the highest level of access permissions. Generally, a medical practitioner may be the page owner for their own profile page and for procedures for which they have primary responsibility. Pages associated with facilities, medical equipment, or other entities may variably be owned by an assigned medical practitioner. Non-owners may have distinct levels of access to pages depending on the configured permissions. Permissions may be granted by the page owner or by another user that has appropriate permissions to assign or relinquish permissions to other users.

Based on different connections available to different users 155, the collaborative medical platform 140 enables a personalized experience for each user 155. For example, upon logging into the collaborative medical platform 140, a user 155 may be presented with personalized interfaces that relate to their connections to other users 155, medical cases, videos, presentations, or other content hosted by the collaborative medical platform 140.

An interface management module 215 manages content associated with various interfaces hosted by the collaborative medical platform 140 and accessible via the client devices 150. As described above, the interface management module 215 may manage pages associated with the various entities managed by the collaborative medical platform 140 including users 155 (which may include medical practitioners, patients, administrators, etc.), medical cases associated with procedures, facilities, medical equipment 160, files or media content, events (e.g., conferences), presentations, training modules, or other data objects. Access to different pages by a specific user 155 may be dependent on that user's connections and permissions configured in the connection graph store 255. Furthermore, patient data may be pseudonymized for viewing by certain other users (dependent on the type of connection and/or permission) such that the patient data cannot be attributed to a specific individual.

A medical case page associated with a medical case may include information organized into preprocedural, intraprocedural, and postprocedural stages. At the preprocedural stage, a medical case page may include information about the patient, the procedure being performed, and the medical practitioner performing the procedure. The interface management module 215 may furthermore provide access to various analytical information (e.g., generated by the analytics module 230 described below) such as risk factors for the patient, experience/expertise of the medical practitioner, outcomes for the type of procedure being planned, or other data. At the intraprocedural stage, the medical case page may provide access to a telepresence session to enable remote collaborators to remotely collaborate with respect to an ongoing procedure. At a postprocedural stage, the medical case page may include information about the patient treatment plan, risk factors, follow up visits, or other postprocedural information.

Some entity pages in the collaborative medical platform 140 may include content feeds to facilitate collaboration between users 155. Content feeds may include various content (e.g., posts) such as text-based commentary, images, video, three-dimensional models, or other multimedia content relating to a medical case. Content may be directly posted to a page associated with a medical case or a post may comprise links to content stored by the collaborative medical platform 140 or on an external server. Posts may be grouped into conversations that hierarchically track the relationships between posts. For example, posts may be made as original posts (which start a new conversation) or as replies to existing posts (which become part of the conversation).

In an example use, a user 155 may invite one or more other users 155 to collaborate on a medical case and thereby gain access to a case page for the medical case. A content feed on the page enables the collaborating users 155 to post to the case page in association with the medical case. The content feed may therefore enable discussion about the procedure to be performed discussion of risk, best practices, or other information that may be useful to the practitioner performing the procedure. Furthermore, the contributing users 155 may post videos or three-dimensional models (or links to content) relating to historical procedures for similarly situated patients. Additionally, contributing users 155 could share links to entity pages associated with past procedures that may be of relevance, to enable a performing medical practitioner to view historical content feeds associated with those procedures. Patient data may optionally be pseudonymized when shared with other users (dependent on the type of connection and/or permission) such that the patient data cannot be attributed to a specific individual.

Content feeds may furthermore be utilized in relation to an ongoing procedure during a real-time telepresence session as discussed in further detail below. Here, a content feed may be presented as a real-time chat window that enables contributors to comment during a procedure, share video, images, or other media, provide links to relevant resources, or otherwise contribute content during the course of procedure.

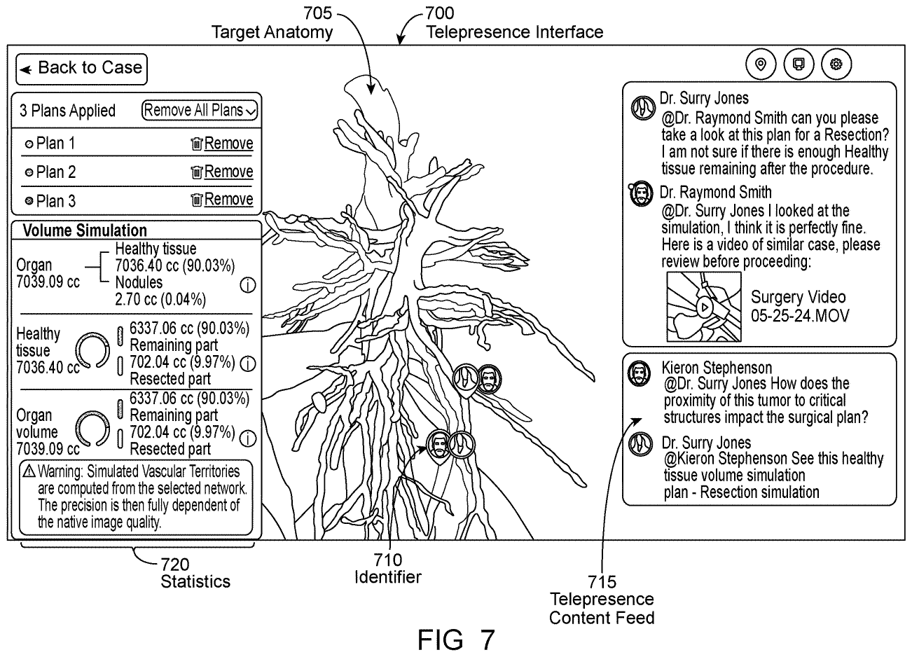

In a postprocedural stage, a content feed may be utilized by contributors to discuss postprocedural treatments, patient recovery, risk management, or other information relevant to patient recovery. Examples of content feeds are provided in FIG. 7 which are described in further detail below.

The medical intelligence module 220 generates medical intelligence data that may be automatically added to content feeds or otherwise made available in the context of the collaborative medical platform 140. For example, the medical intelligence module 220 may automatically contribute posts to a content feed for a medical case that an artificial intelligence agent infers is relevant. Artificially generated posts may mimic posts provided by human contributors and may include text-based commentary, multimedia, links, etc. Medical intelligence data may be generated during a preprocedural stage, during a procedure, or during a postprocedural stage.

In an example implementation, the medical intelligence module 220 may include one or more machine-learned models trained to generate content that the models infer to be relevant to a particular medical case or more generally relevant to a user 155. In one implementation, the machine learned model generates an embedding for a medical case based on descriptive information about a medical procedure, characteristics of the patient on whom the medical procedure is to be performed, characteristics of medical practitioner performing the procedure, posts in the content feed, or other information available in the collaborative medical platform 140. The medical intelligence module 220 determines measures of similarity (e.g., cosine similarity, dot product) between the embedding for the medical case and embeddings for other content available in the collaborative medical platform 140 and that can be included in automated posts. The medical intelligence module 220 may then generate posts and/or select content for posts based on similarities of the embeddings. The medical intelligence module 220 may furthermore employ various Large Language Models (LLMs) to analyze text-based content associated with a medical case and artificially generate relevant natural language content for the content feed. Machine learning models may furthermore include one or more neural networks (such as convolutional neural network (CNN), artificial neural network (ANN), residual neural network (ResNet), or recurrent neural network (RNN)), regression-based models, generative models, or other type of machine-learned model capable of achieving the functions described herein.

In an example use case, the medical intelligence module 220 may identify one or more historical medical cases that are similar to a current medical case and automatically generate a link to a case page for the related case. A medical practitioner may then view videos, models, or other recorded data associated with the related medical case to help the practitioner prepare for a procedure. In other examples, the medical intelligence module 220 may automatically respond to a question posed by a user in the content feed. For example, the medical intelligence module 220 may operate like a chatbot that intelligently responds to text-based queries. In further embodiment, the medical intelligence module 220 may generate a recommendation to invite a specific medical practitioner to collaborate on a medical case based on relevant expertise and experience. A user may then select to invite the recommended collaborator to collaborate on the medical case based on the artificially generated recommendation.

The telepresence module 225 facilitates a telepresence session during a procedure. The telepresence session may be joined by one or more collaborators that have been invited to collaborate on the medical case and enable the other users 155 to remotely access video, telemetry data from one or more medical instruments, or other real-time data captured during a medical procedure. As described above, a content feed may also be displayed in association with the telepresence session to enable contributors to comment or share multimedia or links relevant to the procedure.

The telepresence module 225 may furthermore enable contributors to provide real-time annotations on images, video, three-dimensional models, or other visual content of anatomy relevant to an ongoing procedure. For example, a contributor may mark locations in the visual content in association with provided comments. The telepresence module 225 may furthermore enable contributors to add overlaid drawings, highlighting, or other visual indicators during an ongoing telepresence session.

In an embodiment, the telepresence module 225 may enable remote contributors to take control of medical equipment 160. For example, a remote contributor may access a control interface that provides control elements for controlling a position or orientation of a camera, controlling a robotic arm, setting a configuration of a sensing device, or performing other control functions of medical equipment.

Upon completing a procedure that is captured by the telepresence module 225, the recorded video, telemetry data, content feed, annotations, and other captured data may be stored in association with the procedure. This information may be later accessed by users 155 of the collaborative medical platform 140 (with appropriate permissions) and/or may be utilized by the medical intelligence module 220 to further train machine learning models and/or generate inferences.

The analytics module 230 facilitates generation of various statistics, metrics, or other analytics associated with information stored in the collaborative medical platform 140. Analytics may generally be created based on a set of filtering parameters that yield some subset of data records for aggregating, and a combining function that specifies how the filtered data should be combined. The filtering parameters may filter medical procedure data based on data fields such as patient data, medical practitioner data, facility, procedure type, medical equipment used, etc. The combining function may comprise, for example, an averaging function, a median function, a histogram function, or other function. A specific analytics function may result in a single output value or a series of values over one or more dimensions. Series outputs may be visually represented in a table, chart, graph, or other visual output.

For example, the analytics module 230 may generate metrics describing an average length of time for a specific medical practitioner or a group of medical practitioners to complete a medical procedure. Average times for various procedures performed by the same medical practitioner or group of practitioners may be presented together with similar metrics for other medical practitioners for comparison purposes. In another example, the analytics module 230 may generate metrics describing a number of times a medical practitioner has historically performed a specific type of medical procedure. Such counts could be further aggregated to indicate percentages that reflect how many times a medical practitioner has performed each different type of medical procedure out of a total number of procedures performed.

In further embodiments, the analytics module 230 may generate analytics based on interactions of the medical practitioner in the collaborative medical platform 140. For example, statistics can be derived based on counts of posts, comments, or other content contributed by a medical practitioner to the collaborative medical platform 140. Such analytics may be expressed in terms of counts of interactions, frequency of interactions, or other aggregations. These analytics could furthermore be separately aggregated based on whether interactions relate to preprocedural, intra-procedural, or post-procedural phases of procedures.

In an embodiment, the analytics module 230 may generate analytics based on specific filtering and/or combining functions specified by a user 155 of the collaborative medical platform 140. Additionally, the analytics module 230 may include various preset analytics that may be generated without necessarily receiving specific user inputs. Furthermore, in some embodiments, the medical intelligence module 220 may automatically generate analytics that it infers will be relevant to a specific user 155.

In some embodiments, the analytics module 230 may generate analytics based on any aspects of the collective case data including preprocedural data, telepresence sessions data (including recorded video, telemetry data, in-session content feed data, etc.), and postprocedural data. Analysis associated with telepresence session data may include performing various video processing, content recognition, or other advanced image processing techniques to extract useful information from videos. Furthermore, the analytics module 230 may leverage various medical intelligence data generated from the medical intelligence module 220 to generate analytics.

The practitioner education module 235 manages and stores training data for medical practitioners associated with medical procedures. In various embodiments, the training data comprises reference content items, with each reference content item including descriptive information about a medical procedure. Reference content items maintained by the practitioner education module 235 may include training videos relating to performing medical procedures, articles about medical procedures, articles about patient diagnosis, articles about courses of treatment for a patient recovering from a medical procedure, best practices for a medical procedure, training manuals for medical procedures, instructional material for one or more medical instruments used in a medical procedure, digital training modules, webinars, audio data about a medical procedure (e.g., a podcast about a medical procedure) or other information for training medical practitioners in relation to medical procedures. A reference content item has one or more attributes providing descriptive information about the reference content item. For example, an attribute of a reference content item identifies one or more types of medical procedures associated with the reference content item, allowing the practitioner education module 235 to identify relationships between different reference content items and different types of medical procedures. Other example attributes of a reference content item include: one or more medical practitioners associated with the reference content item (e.g., a medical practitioner who performed a medical procedure associated with the reference content item), a location associated with the reference content item (e.g., a location where a medical procedure associated with the reference content item was performed), a time associated with the reference content item (e.g., a time when the medical procedure associated with the reference content item was performed), one or more medical instruments associated with the reference content item (e.g., one or more medical instruments used in a medical procedure associated with the reference content item), a format of the reference content item (e.g., audio, video, text), or other information describing the reference content item. Reference content items may be locally stored by the collaborative medical platform 140 (e.g., in the video library 250 or another storage device) or retrieved from one or more third-party servers 170 in various embodiments.

Additionally, one or more reference content items comprise reference cases, which are cases having a previously completed medical procedure that a medical practitioner who performed the previously completed medical procedure selected to be available to other medical practitioners. For a reference case, the practitioner education module 235 stores video, telemetry data from one or more medical instruments, or other data captured by the collaborative medical platform 140 during performance of the previously completed medical procedure. In various embodiments, a reference content item comprising a reference case includes a content feed including comments or other data obtained by the collaborative medical platform 140 from contributors during the previously completed medical procedure. The practitioner education module 235 pseudonymizes patient data in a reference case to prevent the reference case from including patient data capable of being attributed to a specific patient. In some embodiments, the pseudonymized patient data in a reference case identifies ranges for one or more types of patient data to provide relevant information about a patient on whom the previously completed medical procedure was performed to another medical practitioner while preventing a specific patient on whom the previously completed medical procedure was performed from being identifiable.

The practitioner education module 235 receives feedback from medical practitioners to whom a reference content item was presented and stores the received feedback in association with the reference content items. For example, the practitioner education module 235 presents a reference content item to a medical practitioner via an interface including one or more feedback elements presented after the reference content item. In some embodiments, the feedback comprises a rating of the reference content items, such as a numerical value within a range. Alternatively, the feedback comprises an indication of satisfaction with the reference content item or an indication of dissatisfaction with the reference comment item. The practitioner education module 235 may maintain an average rating received from medical practitioners for the reference content item or may maintain an aggregated number of indications of satisfaction received for the reference content item (or an aggregated number of indications of dissatisfaction received for the reference content item). Feedback received from medical practitioners, or derived from feedback received from medical practitioners, is stored as an attribute of the reference content item in various embodiments. An attribute of a reference content item may be an indication of positive feedback for the reference content item, which may be stored in response to an average rating of the reference content item equaling or exceeding a threshold value or may be stored in response to a ratio of aggregated number of indications of satisfaction with the reference content item to aggregated number of indications of dissatisfaction with the reference content item equaling or exceeding a threshold value.

In an embodiment, the practitioner education module 235 automatically recommends reference content items, such as reference cases, training modules, or other training materials, to medical practitioners based on monitored performance of a medical practitioner, which may be compared against various averages, metrics, other standards. For example, the practitioner education module 235 may detect that a particular medical practitioner generally performs a specific type of medical procedure in a timeframe that is significantly higher or lower than the global average. This may suggest that the medical practitioner is employing non-standard procedures and the practitioner education module 235. The practitioner education module 235 may detect these discrepancies and recommend one or more reference content items related to the procedure. In some embodiments, one or more machine learning models applied by the practitioner education module 235 may infer reference content items to recommend to a medical practitioner. For example, a machine learning model may be trained to infer, based on characteristics and performance history of a medical practitioner, that a specific reference content item includes information predicted to enable the medical practitioner to improve performance in a manner that increases efficiency and/or improves patient outcomes.

The practitioner education module 235 may use statistics, metrics, or other analytics generated by the analytics module 230 for a medical procedure performed by a medical practitioner to determine whether to present one or more reference content items to the medical practitioner after the medical procedure has been performed. Presenting a reference content item to a medical practitioner after completion of a medical procedure provides the medical practitioner with information for improving or refining subsequent performance of a type of the medical procedure that was completed. In various embodiments, in response to a metric determined for a medical procedure performed by the medical practitioner satisfying one or more criteria, the practitioner education module 235 selects and presents one or more reference content items to the medical practitioner, as further described below. For example, the practitioner education module 235 compares a length of time for the medical practitioner to complete a medical procedure from the analytics module 230 to an average length of time to complete the type of medical procedure (or to a benchmark length of time to complete the type of medical procedure) and presents one or more selected reference content items to the medical practitioner in response to the length of time for the medical practitioner to complete the medical procedure exceeding the average length of time to complete the type of medical procedure (or exceeding the benchmark length of time to complete the type of medical procedure).

Similarly, the practitioner education module 235 may base presentation of one or more reference content items to the medical practitioner on telemetry data captured by the telepresence module 225 during a medical procedure performed by the medical practitioner. In various embodiments, the practitioner education module 235 selects and presents one or more reference content items to a medical practitioner in response to telemetry data captured during a medical procedure satisfying one or more conditions. For example, in response to determining telemetry data captured during the medical procedure includes data indicating one or more specific patterns of movement of a portion of medical equipment 160 during the medical procedure, the practitioner education module 235 selects and presents one or more reference content items associated with movement of the portion of medical equipment 160 to the medical practitioner. As another example, in response to determining telemetry data captured during the medical procedure identifies a specific configuration of a medical robot (or other piece of medical equipment 160), the practitioner education module 235 selects and presents one or more reference content items associated with the specific configuration of the medical robot or selects and presents one or more reference content items identifying an alternative configuration of the medical robot (or other piece of medical equipment 160). A selected reference content item may describe alternative techniques for moving the medical equipment 160 during a type of medical procedure or provide additional details about how to move the medical equipment 160. In another example, in response to telemetry data indicating the medical practitioner used a specific piece of medical equipment 160 during the medical procedure, the practitioner education module 235 selects and presents one or more reference content items relevant to an alternative piece of medical equipment to the medical practitioner for subsequent use in similar medical procedures. In another example, the telemetry data, or stored data describing a medical procedure, identifies pieces of medical equipment used by the medical practitioner in a type of medical procedure, and the practitioner education module 235 compares medical equipment used by the medical practitioner for the medical procedure to alternative pieces of medical equipment used by other medical practitioners in the type of medical procedure. The practitioner education module 235 selects a reference content item describing use of an alternative piece of medical equipment in the type of medical procedure. For example, the practitioner education module 235 selects a reference content item describing use of a more cost effective alternative piece of medical equipment in the type of medical procedure, providing the medical practitioner with information for reducing a cost of performing the medical procedure. As another example, the practitioner education module 235 selects a reference content item describing use of an alternative piece of medical equipment that reduces an amount of time to complete the type of medical procedure. The practitioner education module 235 may select reference content items describing use of an alternative piece of medical equipment based on one or more metrics determined for medical procedures in which the alternative piece of medical equipment was used. In embodiments where telemetry data captured during the medical procedure includes vital signs of the patient captured by one or more monitoring devices, the practitioner education module 235 selects and presents one or more reference content items related to the medical procedure in response to determining one or more vital signs of the patient satisfied one or more criteria (e.g., were within a specific range, were outside of a specific range, etc.) during the medical procedure. The practitioner education module 235 may maintain different criteria for different vital signs or different telemetry data for different types of medical procedures to further refine when reference content items are selected and presented to a medical practitioner.

Additionally or alternatively, the practitioner education module 235 selects one or more reference content items for a medical practitioner based on data in the user profile of the medical practitioner and presents one or more of the selected reference content items to the medical practitioner. The user profile of a medical practitioner includes characteristics describing the medical practitioner, and the practitioner education module 235 uses one or more of these characteristics to determine when to select and present one or more reference content items to the medical practitioner. For example, a characteristic of a medical practitioner identifies an area of specialization of the medical practitioner, and the practitioner education module 235 selects one or more reference content items having an attribute identifying a medical procedure within the area of specialization of the medical practitioner. As another example, a characteristic of the medical practitioner in the user profile identifies a medical procedure scheduled to be performed at a specific time by the medical practitioner, and the practitioner education module 235 selects one or more reference content items having an attribute identifying a type of the medical procedure scheduled to be performed by the medical practitioner.

In some embodiments, the practitioner education module 235 accounts for connections between additional medical practitioners and the medical practitioner in the connection graph store 255 when selecting a reference content item. For example, the practitioner education module 235 accounts for additional medical practitioners collaborating with the medical practitioner on a medical procedure when selecting a reference content item for the medical practitioner. As an example, the collaborative medical platform 140 selects a reference content item associated with a type of medical procedure matching a medical procedure scheduled to be performed by an additional medical practitioner identified by the connection graph store 255 as a collaborator on the medical procedure with the medical practitioner. The reference content item may be selected based on a user profile of an additional user identified as a collaborator with the medical practitioner on a medical procedure. For example, the additional medical practitioner is a medical practitioner supervising the medical practitioner on the medical procedure. As another example, the additional medical practitioner is a medical practitioner performing a portion of the medical procedure with the medical practitioner. This allows a reference content item selected for the medical practitioner to account for potential preferences or techniques employed by the additional medical practitioner collaborating with the medical practitioner.

The practitioner education module 235 presents one or more reference content items selected for a medical practitioner to the medical practitioner through one or more interfaces. For example, a practitioner dashboard includes a section that identifies one or more reference content items to a medical practitioner. As another example, an education interface presents one or more selected reference content items to the medical practitioner.

The practitioner education module 235 may apply one or more trained machine learning models to characteristics of the medical practitioner in a user profile and to attributes of reference content items to select one or more reference content items for presentation to the medical practitioner. As further described above, each reference content item has one or more attributes. Example attributes of a reference content item include: a type of medical procedure associated with the reference content item, one or more medical practitioners associated with the reference content item, a location where the medical procedure was performed (e.g., a geographic location, an identifier of a medical facility), a format of the reference content item (e.g., text data, audio data, video data, etc.), feedback about the reference content item from or more medical practitioners (e.g., a rating, an amount of positive feedback received for the reference content item, etc.), or other descriptive information. A user profile maintained by the collaborative medical platform 140 for a medical practitioner includes characteristics of the medical practitioner. Example characteristics of a medical practitioner include: an area of specialization of the medical practitioner, types of prior medical procedures performed by the medical practitioner, medical procedures scheduled to be performed by the medical practitioner, a location where the medical practitioner performs medical procedures (e.g., a geographic location, an identifier of a medical facility, etc.), collaborators connected to the medical practitioner via the connection graph, or other descriptive information about the medical practitioner. The practitioner education module 235 trains one or more machine-learning models to select one or more reference content items for a medical practitioner based on attributes of reference content items and characteristics of the medical practitioner in various embodiments. Example machine learning models include regression models, support vector machines, naïve Bayes, decision trees, k nearest neighbors, random forest, boosting algorithms, k-means, and hierarchical clustering. The machine learning models may also include neural networks, such as perceptrons, multilayer perceptrons, convolutional neural networks, recurrent neural networks, sequence-to-sequence models, generative adversarial networks, or transformers, while other types of machine learning models may additionally or alternatively be trained or applied by the practitioner education module 235 in various embodiments.

For example, to train a machine learning model to select one or more reference content items, the practitioner education module 235 generates a set of training examples, with each training example including characteristics of a medical practitioner and attributes of a reference content item and having a label indicating whether the medical practitioner in the training example accessed the reference content item included in the training example (or indicating whether the medical practitioner in the training example provided positive feedback for the reference content item included in the training example).

Applying the machine learning model to a training example generates a predicted likelihood of the medical practitioner in the training example accessing the reference content item in the training example (or a predicted likelihood of the medical practitioner in the training example providing positive feedback for the reference content item included in the training example). For each training example to which the practitioner education module 235 applies the machine learning model, the practitioner education module 235 generates a score for the machine learning model comprising an error term based on the label applied to the training example and the predicted likelihood of the medical practitioner in the training example accessing the reference content item in the training example (or a predicted likelihood of the medical practitioner in the training example providing positive feedback for the reference content item included in the training example). The error term, and accordingly the score, is larger when a difference between the label applied to the training example and the predicted likelihood of the medical practitioner in the training example accessing the reference content item in the training example (or the predicted likelihood of the medical practitioner in the training example providing positive feedback for the reference content item included in the training example) is larger and is smaller when the difference between label applied to the training example and the predicted likelihood of the medical practitioner in the training example accessing the reference content item in the training example (or the predicted likelihood of the medical practitioner in the training example providing positive feedback for the reference content item included in the training example) is smaller. In various embodiments, the practitioner education module 235 generates the score for the machine learning model applied to a training example using a loss function based on the difference between the label applied to the training example and the predicted likelihood of the medical practitioner in the training example accessing the reference content item in the training example (or the predicted likelihood of the medical practitioner in the training example providing positive feedback for the reference content item included in the training example). Example loss functions include a mean square error function, a mean absolute error function, a hinge loss function, and a cross-entropy loss function.

The practitioner education module 235 backpropagates the error term to update a set of parameters comprising the machine learning model and stops backpropagation in response to the score, or to a loss function, satisfying one or more criteria. For example, the practitioner education module 235 backpropagates the score for the machine learning model through the layers of the machine learning model to update parameters of the machine learning model until the score has less than a threshold value. For example, the practitioner education module 235 uses gradient descent to update the set of parameters comprising the machine learning model. The practitioner education module 235 stores the trained machine learning model for application to characteristics of one or more medical practitioners and to attributes of one or more reference content items. In some embodiments, the practitioner education module 235 trains and maintains different machine learning models that each use different combinations of attributes of a reference content item and characteristics of a medical practitioner.

In various embodiments, to select a reference content item for presentation to a medical practitioner, the practitioner education module 235 selects a set of reference content items each having an attribute that at least partially matches a characteristic in the user profile of the medical practitioner. The practitioner education module 235 applies the machine learning model to each combination of the medical practitioner and a reference content item of the set, generating a predicted likelihood of the medical practitioner accessing the reference content item of the set (or providing positive feedback for the reference content item of the set) for each reference content item of the set. Based on the predicted likelihoods of being accessed by the medical practitioner (or of receiving positive feedback from the medical practitioner), the practitioner education module 235 selects one or more reference content items for presentation to the medical practitioner. For example, the practitioner education module 235 ranks the reference content items based on the predicted likelihoods of being accessed by the medical practitioner (of or receiving positive feedback from the medical practitioner) and selects one or more reference content items having at least a threshold position in the ranking. As another example, the practitioner education module 235 selects reference content items having at least a threshold predicted likelihood of being accessed by the medical practitioner (or having at least a threshold predicted probability of receiving positive feedback from the medical practitioner).

The practitioner education module 235 may alternatively select a reference content item for a medical practitioner based on scores determined for various reference content items. In various embodiments, a score for a reference content item is based on an amount of attributes of the reference content item matching a characteristic of the medical practitioner. For example, the practitioner education module 235 determines a score for a reference content item as a sum of attributes of the reference content item matching a characteristic of the medical practitioner, so reference content items with a larger number of attributes matching characteristics of the medical practitioner have higher scores and reference content items with a smaller number of attributes matching characteristics of the medical practitioner have lower scores. The practitioner education module 235 ranks reference content items based on their scores and selects one or more reference content items having at least a threshold position in the ranking for presentation to the medical practitioner. Alternatively, the practitioner education module 235 selects reference content items having at least a threshold score for presentation to the medical practitioner.

In various embodiments, the practitioner education module 235 assigns different weights to different characteristics of the medical practitioner and uses the weights when determining a score for a reference content item. For example, a score for a reference content item comprises a sum of weights assigned to characteristics of the medical practitioner that match an attribute of the reference content item. This allows attributes of a reference content item matching different characteristics of the medical practitioner to differently contribute to the score for the reference content item. For example, a characteristic of a contributor connected to the medical practitioner has a higher weight than other characteristics of the medical practitioner, so reference content items with an attribute matching a contributor connected to the medical practitioner have higher scores than other reference content items. The practitioner education module 235 may dynamically modify weights assigned to characteristics of a medical practitioner in some embodiments. For example, within a threshold amount of time before a specific type of upcoming medical procedure scheduled to be performed by the medical practitioner, the practitioner education module 235 increases a weight of a characteristic identifying the specific type of medical procedure and increases a weight of a characteristic identifying a collaborator with the medical practitioner on the upcoming medical procedure. In the preceding example, reference content items with attributes identifying the specific type of medical procedure and the collaborator will have higher scores than other reference content items, as reference content items relating the specific medical procure and the collaborator are more likely to be relevant to the medical practitioner prior to the upcoming medical procedure.

Alternatively or additionally, one or more machine learning models applied by the practitioner education module 235 to select a reference content item are nearest neighbor models applied to embeddings corresponding to reference content items and to characteristics of the medical practitioner. As further described above, attributes of a reference content item include: a type of medical procedure associated with the reference content item, one or more medical practitioners associated with the reference content item, a location where the medical procedure was performed (e.g., a geographic location, an identifier of a medical facility), a format of the reference content item (e.g., text data, audio data, video data, etc.), feedback about the reference content item from or more medical practitioners (e.g., a rating, an amount of positive feedback received for the reference content item, etc.), or other descriptive information. Example characteristics of a medical practitioner include: an area of specialization of the medical practitioner, types of prior medical procedures performed by the medical practitioner, medical procedures scheduled to be performed by the medical practitioner, a location where the medical practitioner performs medical procedures (e.g., a geographic location, an identifier of a medical facility, etc.), collaborators connected to the medical practitioner via the connection graph, or other descriptive information about the medical practitioner.

In some embodiments, the practitioner education module 235 applies a nearest neighbor model to an embedding of the medical practitioner that determines a distance (or a measure of similarity) in a latent space between the embedding of the medical practitioner and embeddings for various reference content items. For example, the nearest neighbor model determines a Euclidean distance between the embedding of the medical practitioner and embeddings for reference content items. Based on the distances, the nearest neighbor model ranks reference content items by the distances (or measures of similarity) of their corresponding embeddings to the embedding of the medical practitioner and selects one or more reference content items having a threshold position in the ranking, so the selected one or more reference content items have embeddings nearest to the embedding of the medical practitioner. Alternatively, the nearest neighbor model selects one or more reference content items having less than a threshold distance from the embedding for the medical practitioner. Alternatively, the practitioner education module 235 generates an embedding for a medical procedure performed by the medical practitioner and selects one or more reference content items based on distances between the embedding for the medical procedure and embeddings for reference content items, as further described above.

Further, in some embodiments, the practitioner education module 235 generates embeddings for different medical practitioners based on characteristics of the medical practitioners, as further described above. The practitioner education module 235 determines distances between an embedding for a medical practitioner and embeddings for additional medical practitioners. For example, the practitioner education module 235 determines Euclidean distances between the embedding for the medical practitioner and embeddings for multiple additional medical practitioners. Based on the distances (or measure of similarity), the practitioner education module 235 selects a set of additional medical practitioners. For example, the practitioner education module 235 selects additional medical practitioners with embeddings within a threshold distance of the embedding of the medical practitioner. As another example, the practitioner education module 235 ranks additional medical practitioners based on distances between their embeddings and the embedding of the medical practitioner and selects additional medical practitioners having at least a threshold position in the ranking. The practitioner education module 235 selects one or more reference content items presented to one or more of the selected additional medical practitioners for presentation to the medical practitioner. Such embodiments allow the practitioner education module 235 to leverage similarity between various medical practitioners to select reference content items for presentation to the medical practitioner.

The practitioner education module 235 accounts for attributes of reference content items a medical practitioner accessed over time (or of reference content items with which the medical practitioner provided positive feedback) when selecting one or more reference content items for presentation in various embodiments. For example, some medical practitioners prefer to view video reference content items, while other medical practitioners prefer to listen to audio reference content items. The practitioner education module 235 accounts for preferences of different medical practitioners for different formats of reference content items when selecting a reference content item for a medical practitioner so a reference content item selected for the medical practitioner has the medical practitioner's preferred format. In various embodiments, the practitioner education module 235 maintains a log of reference content items accessed by a medical practitioner over time. The practitioner education module 235 applies a format selection model to the log of reference content items to determine a preferred format of reference content items accessed by the medical practitioner based on statistical analysis of formats of reference content items the medical practitioner previously accessed (or for which the practitioner education module 235 received positive feedback from the medical practitioner). For example, based on frequencies with which the medical practitioner accessed different reference content items with different formats over time, as well as other attributes of reference content items or characteristics of the medical practitioner, a format selection model determines a preferred format of reference content item for the medical practitioner. In various embodiments, the practitioner education module 235 selects a reference content item having the preferred format (e.g., audio, video, text) for presentation to the medical practitioner. For example, the practitioner education module 235 ranks reference content items based on corresponding likelihoods of being accessed by the medical practitioner (or likelihoods of receiving positive feedback from the medical practitioner), as further described above, and selects a reference content item having the preferred format of the medical practitioner with a highest position in the ranking. Alternatively, the practitioner education module 235 selects a set of reference content items that each have the preferred format of the medical practitioner and have an attribute that at least partially matches a characteristic in the user profile of the medical practitioner (e.g., a type of medical procedure matching a type of medical procedure in the user profile of the medical practitioner). The practitioner education module 235 selects one or more reference content items of the set based on their likelihoods of being accessed by the medical practitioner (or likelihoods of receiving positive feedback from the medical practitioner), as further described above.

In various embodiments, the practitioner education module 235 also leverages a log of reference content items accessed by a medical practitioner to determine a time for presenting one or more reference content items to the medical practitioner. For example, the practitioner education module 235 stores an identifier of a reference content item accessed by the medical practitioner and stores a time when the medical practitioner accessed the reference content item in association with the identifier of the reference content item. The practitioner education module 235 also retrieves a procedure time for medical procedures performed by the medical practitioner from the medical practitioner's user profile. For each medical procedure the medical practitioner performed, the practitioner education module 235 determines a time interval between the procedure time for the medical procedure and a time before the medical procedure when the medical practitioner accessed a reference content item associated with a type of the medical procedure. By applying one or more timing machine learning models to time intervals between procedure times for different medical procedures and times before different medical procedures when the medical practitioner accessed one or more reference content items associated with the type of the medical procedure, the practitioner education module 235 determines a preferred time interval before a medical procedure for the medical practitioner to access a reference content item relevant to the medical procedure.

The practitioner education module 235 may determine a preferred time interval before different types of medical procedure for the medical practitioner in various embodiments. For example, the practitioner education module 235 identifies prior medical procedures performed by the medical practitioner having a specific type and determines a time interval between a procedure time for each identified prior medical procedure and a time before an identified medical procedure when the medical practitioner accessed a reference content item associated with the specific type of the medical procedure. The practitioner education module 235 applies the one or more timing machine learning models to time intervals between procedure times for different medical procedures with the specific type and times before the medical procedures with the specific type when the medical practitioner accessed one or more reference content items associated with the specific type to determine a type-specific preferred time interval before the specific type of medical procedure when the medical practitioner accesses a reference content item relevant to the specific type of medical procedure. The practitioner education module 235 determines type-specific preferred time intervals for different specific types of medical procedures for the medical practitioner and stores the type-specific preferred time intervals in association with the medical practitioner and with a corresponding specific type of medical procedure. This allows the practitioner education module 235 to maintain a record of type-specific preferred time intervals for presenting reference content items to the medical practitioner before performing different types of medical procedures.

Based on the preferred time interval before a medical procedure, the practitioner education module 235 determines when to present a selected reference content item to the medical practitioner relative to a medical procedure to be performed by the medical practitioner. For example, the practitioner education module 235 identifies a procedure time when the medical practitioner is scheduled to perform a medical procedure from the medical practitioner's user profile and initially presents a selected reference content item to the medical practitioner based on the preferred time interval and the procedure time. In various embodiments, the practitioner education module 235 determines a time when the reference content item is initially presented to the medical practitioner by subtracting the preferred time interval from the procedure time of the medical procedure. As an example, the preferred time interval for the medical practitioner is 12 hours prior to a procedure time of a medical procedure and the medical practitioner is scheduled to perform the medical procedure at 8 AM, so the practitioner education module 235 presents a reference content item relevant to the medical procedure at 8 PM the day before the medical procedure. This allows the practitioner education module 235 to proactively present a reference content item to a medical practitioner at a time when the medical practitioner is most likely to access the reference content item.

The practitioner education module 235 may use one or more statistics, metrics, or other analytics generated by the analytics module 230 for a medical procedure performed by the medical practitioner in a medical case to determine whether to subsequently use the medical case in which the medical procedure as a reference case. For example, if one or more metrics generated by the analytics module 230 indicate a positive patient outcome from the medical procedure (e.g., a metric has a specific value, a metric is within a range of values, a metric is less than a threshold, a metric is greater than a threshold, etc.), the patient education module 235 presents a message to the medical practitioner to generate a reference case based on the medical case in which the medical procedure was performed. In response to receiving an authorization from the medical practitioner to generate the reference case, the patient education module 235 stores video, telemetry data from one or more medical instruments, or other data captured during performance of the medical procedure, and may store a content feed including comments or other data obtained by the collaborative medical platform 140 during the medical procedure from contributors to the medical procedure. Additionally, the practitioner education module 235 pseudonymizes patient data for the medical procedure when generating the reference case to prevent attribution of patient data in the reference case to a specific patient. In various embodiments, the practitioner education module 235 generates one or more attributes of the reference case from captured or previously stored information describing the medical procedure, while in other embodiments the practitioner education module 235 prompts the medical practitioner to specify one or more attributes of the reference case. This allows the practitioner education module 235 to simplify creation of reference cases to aid in educating medical practitioners about various types of medical procedures.

The presentation module 240 leverages stored information associated with a completed medical procedure to facilitate generation of presentations for education, research, training, or other purposes. Presentations may be in the form of slide decks, posters, videos, animations, or other multimedia content. Presentations may incorporate various multimedia (e.g., video, images, three-dimensional models, and associated metadata), patient record data, medical equipment telemetry data, information from content feeds, analytics, or other information generated and/or stored by the collaborative medical platform 140.

In an embodiment, the presentation module 240 may maintain one or more presentation templates for generating presentations. The template may include pre-formatted content with various information fields that may be automatically populated from a set of records. For example, a practitioner wanting to prepare a presentation relating to a set of recently performed procedures may specify the set of procedures to include in the presentation, and the presentation module 240 may automatically populate the presentation based on the data stored in association with those procedures, pages, with each page associated with one or more types of data about the completed medical procedure. In some embodiments, the presentation module 240 may apply one or more trained machine learned models to automatically generate and/or recommend presentation content that may be of interest to a medical practitioner. In further embodiments, the presentation module 240 may intelligently automatically de-identify patient data included in the presentations.

The presentation module 240 may furthermore include various editing tools for creating, viewing, and editing presentations. For example, the editing tools may enable editing of text, video, images, animations, three-dimensional models, or other content for including in a presentation.

In an embodiment, presentations may be presented through a presentation module 240 directly without data associated with the presentation being exported externally to the collaborative medical platform 140. For example, the presentation module 240 may enable live streamlining of a presentation during a telepresence session to a set of invited attendees. The invited attendees may be limited to users 155 of the collaborative medical platform 140 or may include outside attendees that may gain access via an external link. Sharing presentations in this manner enables practitioners to maintain data privacy and compliance and avoid issues that may arise when externally exporting medical data.

The application integration module 245 manages integration of applications with the collaborative medical platform 140. Applications may be utilized to add additional optional functionality to the collaborative medical platform 140. For example, applications may enable integration with a specific EHR system, scheduling system, or other existing medical system. Applications may furthermore enable users to selectively add specific functionality beyond the core features of the collaborative medical platform 140. The application integration module 245 may allow third parties to create applications that interface with the collaborative medical platform 140 and make these applications available to add.

The application integration module 245 may maintain a catalog of applications capable of interfacing with the collaborative medical platform 140 and may provide interfaces to enable users 155 to selectively add applications for integration. In various embodiments, applications identified by the application integration module 245 have been authorized or approved for installation by an administrator of the collaborative medical platform 140, allowing regulation of the applications capable of executing on the collaborative medical platform 140.

Additionally, the application integration module 245 may include one or more application programming interfaces (API) for an application installed through the application integration module 245. An API for an application provides functionality for exchanging data between the application and one or more components of the collaborative medical platform 140, simplifying data exchange between the application and other portions of the collaborative medical platform 140.

The video library 250 stores videos of various medical procedures, training presentations, simulations, or other medical videos and metadata associated with the video. Examples of metadata associated with video of a medical procedure may include telemetry data of one or more medical instruments received in conjunction with the video, comments or annotations received from one or more medical practitioners through a surgical interface during the medical procedure included in the video, segmentation data that divides the video into temporal segments relating to different step of a procedure, profile information (e.g., age, body mass index, gender, etc.), associated with the patient in the video, or other information supplementing the video. Various reference content items including video data may be stored in the video library 250 for retrieval by the practitioner education module 235 in various embodiments.

The video library 250 may store videos in an indexed database that indexes videos based on various metadata. The video library 250 can then be browsed or searched via a video library interface to identify videos of relevance. The metadata associated with videos may include permissions stored in the connection graph store 255 that controls which users 155 have access to different videos. For example, a video in the video library 250 may be accessible only to users 155 that the video has been expressly shared with or that otherwise has viewing permissions for the video.

The connection graph store 255 comprises a database that stores information describing connections between entities or other objects (e.g., videos or other multimedia) managed by the collaborative medical platform 140. For example, as described above, the connection graph store 255 stores connections between users 155, connections between users 155 and procedures, connections between users 155 and multimedia content or other objects, or other connections between data entities of the collaborative medical platform 140.

The user profile store 260 stores profile data for users 155 of the collaborative medical platform 140. A user profile for a medical practitioner includes descriptive information such as a name of the medical practitioner, contact information for the medical practitioner, credentials or certifications of the medical practitioner, biographical information for the medical practitioner, types of medical procedures capable of being performed by the medical practitioner, medical facilities affiliated with the medical practitioner, operating room preferences (such as patient positioning, equipment setup, preferred instrumentation, typical procedure step order, etc.), equipment configuration preferences (e.g., ergonomic settings for a robot console), or other information describing the medical practitioner. Aspects of the user profile could be inferred using machine learning techniques. For example, a practitioner's preferred instrumentation or step order may be inferred from application of a machine learning model trained to infer such preferences based on observed historical data. Additionally, a user profile for a medical practitioner includes medical procedures performed by or to be performed by the medical practitioner, as well as information describing the medical procedures. For example, the user profile identifies different types of medical procedures to be performed by, or performed by, the medical practitioner, and may include characteristics for each medical procedure (e.g., a length of time to complete the medical procedure, a number of times the medical practitioner performed a type of medical procedure matching the medical procedure, etc.). Further, one or more of the metrics determined by the analytics module 230 for the medical practitioner, as further described above, may be included in the user profile for the medical practitioner.

The patient data store 265 includes a patient profile for each patient associated with medical cases. A patient profile includes characteristics of a corresponding patient, which may be obtained from an electronic health record for the patient or may be provided via input from a medical practitioner. Characteristics of a patient include demographic information about the patient, medical conditions of the patient, medical procedures previously performed by the patient, allergies of the patient, contact information for the patient, current or prior prescriptions for the patient or other medically relevant information about the patient. A patient identifier is associated with a patient profile to uniquely identify the patient profile.

All of the data stored to the collaborative medical platform 140 (or otherwise made available through the collaborative medical platform 140) may be stored, presented, and in some cases restricted in a manner that ensures compliance with various data privacy and protection regulations.

FIGS. 3A-3B illustrate an example practitioner dashboard 300. FIG. 3A shows an upper portion of the dashboard 300 while FIG. 3B shows a lower portion of the dashboard 300 (which may be continuously scrollable). The practitioner dashboard 300 may operate as a home landing page for a medical practitioner upon logging into the collaborative medical platform 140. The practitioner dashboard 300 may include various content sections, at least some of which may be specifically targeted to the practitioner. A search bar 305 enables input of text-based search queries for searching content available in the collaborative medical platform 140 (e.g., case pages, other user pages, videos, presentations, etc.). In response to inputting a search query, a list of results may be displayed with links to content matching the search query. A video promotion section 310 shows a video recently added by the practitioner with user interface tools to enable the practitioner to promote the video by sharing it with other users, create a highlight reel, or view various statistical information about the video. An achievement section 315 presents an achievement relating to use of the collaborative medical platform 140. In this example, the achievement section 315 highlights that the user has recently reached 100 videos and provides links to view the user's videos and access a video library. Other examples of achievements in the achievement section 315 could relate to number of cases managed, time using the platform 140, number of connections, count of frequency of interactions, or other usage achievements. The video library 320 includes video thumbnails, video tags, or other links to enable browsing of videos selected as potentially relevant to the medical practitioner. For example, relevant videos may be selected that relate to past or upcoming procedures associated with the medical practitioner, based on a history of videos viewed by the medical practitioner, based on a practice area or other profile information for the medical practitioner, or other factors. The webinar promotion section 325 includes a promotional banner for an upcoming webinar that will be viewable within the collaborative medical platform 140. The webinar may be identified as being of potential interest to the medical practitioner based on, for example, the subject matter of the webinar, the host of the webinar, or other factors. The shared cases section 330 provides summary information and links to case pages that have been shared with the medical practitioner. Examples of case pages are described in further detail below. The analytics summary 335 includes example analytics associated with the medical practitioner's usage of the collaborative medical platform 140, procedures performed by the medical practitioner, or other analytics data derived from information stored in the collaborative medical platform 140. The analytical data may be presented in one or more visual representations such as a graph or chart. The feedback section 340 provides links to enable the medical practitioner to send feedback to an administrator of the collaborative medical platform 140.

FIGS. 3A-3B illustrate just one example of a practitioner dashboard 300. The types of content presented in the practitioner dashboard 300 may be different for different practitioners and/or may dynamically change over time for the same medical practitioner. Some of the sections may be fixed and always appear upon accessing the dashboard 300 (e.g., the search bar 305, video library 320, shared cases 330, analytics 335, and feedback sections 340), while other sections (e.g., video promotion 310, achievement 315, webinar promotion 325) may be dynamically inserted only in certain contexts. For example, webinar promotions 325 may be presented only when an upcoming webinar deemed to be of sufficient interest is upcoming. Achievements 315 may similarly be displayed only when a relevant achievement has recently been achieved. Furthermore, the dashboard 300 could be customized by the user to display desired sections in a configured order. The various sections 305, 310, 315, 320, 325, 330, 335, 340 when present, may furthermore be presented in different order in different contexts.

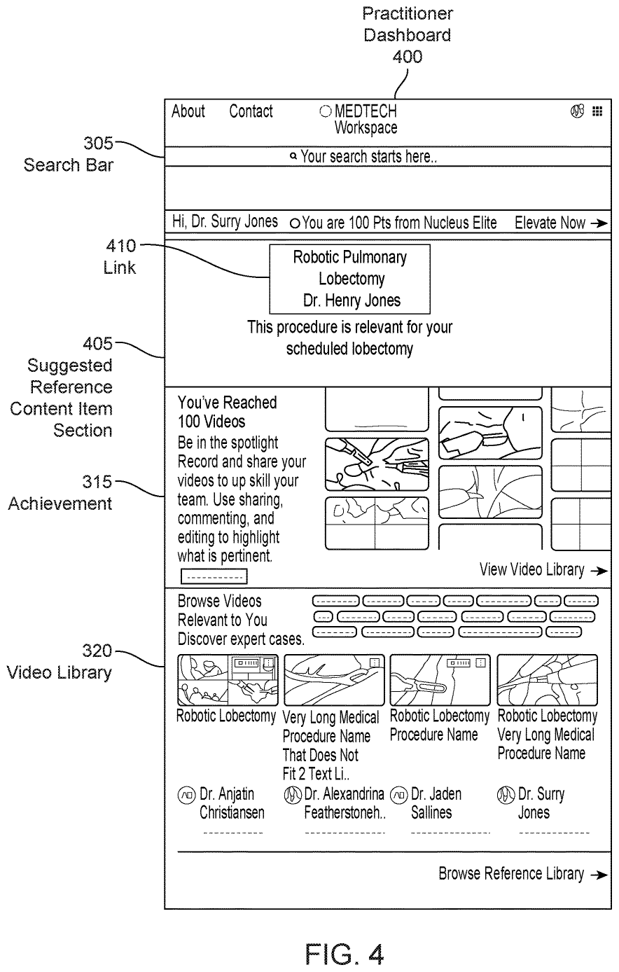

FIG. 4 shows an alternative embodiment of a practitioner dashboard 400. In the example shown by FIG. 4, the practitioner dashboard 400 includes a suggested reference content item section 405 identifying a reference content item and including a link 410 to access the reference content item. The practitioner education module 235 selects the reference content item identified by the suggested reference content item section 404, as further described above in conjunction with FIG. 2. In various embodiments, the suggested reference content item section 405 identifies one or more reasons why the identified reference content item is identified as being of potential interest to the medical practitioner. In the example of FIG. 4, the suggested reference content item section 405 indicates that the identified reference content item is related to a medical procedure that the medical practitioner is scheduled to perform. As another example, the suggested reference content item section 405 indicates the identified reference content item is related to an area of specialization of the medical practitioner.

For purposes of illustration, FIG. 4 shows an example practitioner dashboard 400 where the suggested reference content item section 405 is displayed proximate to a search bar 305. For example, the suggested reference content item section 405 is displayed in a position of the practitioner dashboard 400 below the search bar 305, so the suggested reference content item section 405 is prominently displayed in the practitioner dashboard 400 to increase a likelihood of the medical practitioner selecting the link 410 to the identified reference content item. However, in other embodiments, the practitioner dashboard 400 displays the suggested reference content item section 405 in a different position relative to other sections. Similarly, while FIG. 4 shows an example where the achievement section 315 and the video library section 320 are displayed in conjunction with the suggested reference content item section 405, in other embodiments, different or additional sections are displayed by the practitioner dashboard 400 in conjunction with the suggested reference content item section 405.

In various embodiments, the suggested reference content item section 405 is dynamically inserted into the practitioner dashboard 400 in certain contexts and is not included in the practitioner dashboard 400 in other contexts. For example, the practitioner dashboard 400 displays the suggested reference content item section 405 a determined time interval before a time when a medical practitioner is scheduled to perform a medical procedure. This allows the medical practitioner to more easily identify and access one or more reference content items relevant to the scheduled medical procedure. However, in various embodiments, the practitioner dashboard 400 does not display the suggested reference content item section 405 at times greater than the determined time interval before the time when the medical practitioner is scheduled to perform a medical procedure.

In some embodiments, the collaborative medical platform 140 generates one or more education interfaces, such as an education dashboard. An education interface may additionally or alternatively display the suggested reference content item section 405 to a medical practitioner, providing an additional way for the medical practitioner to access the suggested reference content item. The practitioner dashboard 400 may include an interface element that, when selected by the medical practitioner, causes display of the education interface. The education interface may display information identifying multiple suggested reference content items in some embodiments, allowing the medical practitioner to more easily access a wider range of suggested reference content items.

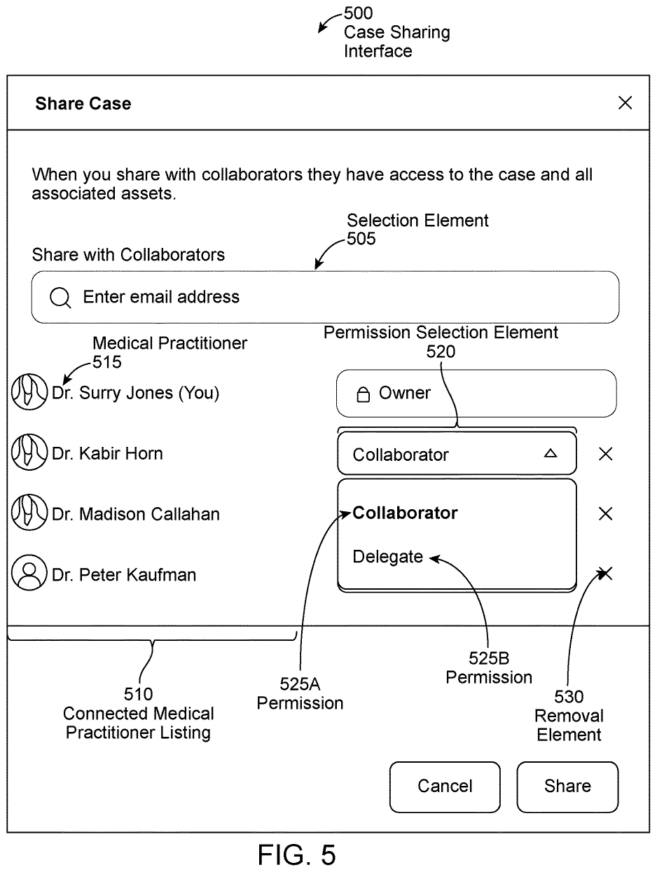

FIG. 5 shows an example embodiment of a case sharing interface 500 for sharing a case with one or more contributors. Adding a contributor to a case may generate a connection between the contributor and the case and between the contributor and the case owner. The case sharing interface 500 includes a selection element 505 for receiving identifying information to identify a desired contributor. For example, the selection element 505 may receive an email address, name, a username, or another identifier of a medical practitioner or other requested contributor. In some embodiments, upon selecting identifying information for a desired contributor, the case sharing interface 500 may display all or a portion of profile data for the requested collaborator to enable the requestor to confirm if the matched profile data is the intended collaborator. The case sharing interface 500 then enables the requestor to confirm or decline selection of a collaborator and interact with a permission selection element 520 to set a desired permission level for the requested collaborator. Here, the permission level may place limits on an invited collaborator's access to data about the case and/or may limit actions the collaborator is permitted to perform in association with the case. In an example embodiment, the permission level may be selected between a “collaborator” level 525A and a “delegate” level 525B.

In response to receiving inputs to select a requested collaborator and set a desired permission level (via the permission selection element 520), the case sharing interface 500 may send an invitation to the requested contributor (e.g., via an email, text message, phone call, portal message, or other communication mechanism) to enable the requested collaborator to accept or decline the request. If the request is accepted, the case sharing interface 500 may add the identifier or other information for the new collaborator to a connected medical practitioner listing 510 that lists the contributors added to the case. For example, the illustrated example shows a connected medical practitioner listing 510 that includes the case owner 515 and three additional contributors that have been added to the case.

The case sharing interface 500 may furthermore enable the case owner to change permission levels of existing contributors in the connected medical practitioner listing 510. Furthermore, the case sharing interface 500 may include removal elements 530 associated with each contributor in the connected medical practitioner listing 510 that enables removal of a contributor from the case. Selection of a removal element 530 may remove the stored connection in between the practitioner and the case, such that the practitioner no longer has access to the case.

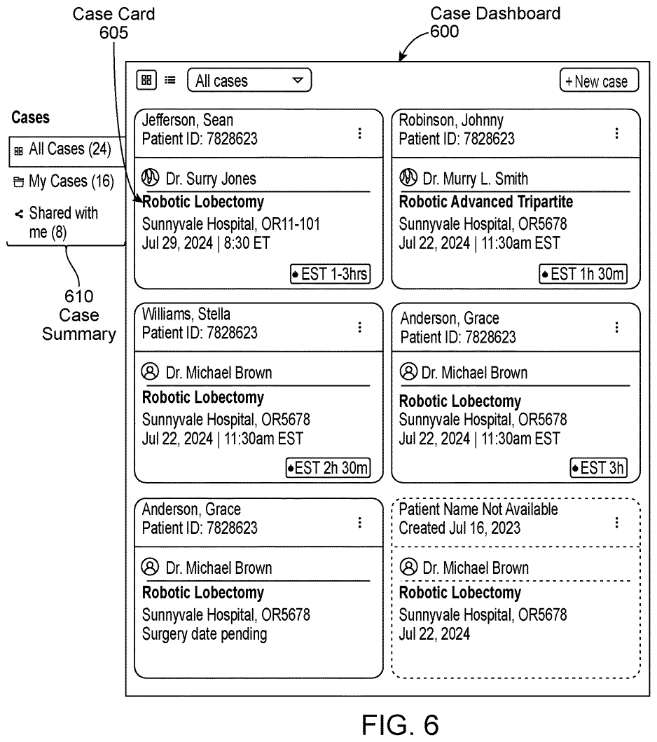

FIG. 6 is an example embodiment of a case dashboard 600 for a medical practitioner. The case dashboard 600 enables access to cases owned by the medical practitioner and cases shared with the medical practitioner by other users 155 as indicated in the case summary 610. In this example, the case dashboard 600 is organized as a set of case cards 605 that each graphically show a summary of a case. Selecting a case card 605 links to a case page for the case. In alternative embodiments, the dashboard 600 may be presented in a list view or other view without necessarily presenting case cards 605 in the visual form shown in FIG. 6.

FIG. 7 shows an example of a telepresence interface 700 associated with a telepresence session that may take place during an actual procedure or during a simulated procedure. Alternatively, the telepresence session may be utilized for live planning purposes without necessarily performing or simulating a procedure. In this example, the telepresence interface 700 displays a three-dimensional model of a target anatomy 705 associated with the procedure. The model may include annotated comments that may be obtained during the telepresence session or that were added in a preprocedural stage. Alternatively, the telepresence interface 700 may include a view of real-time video or images associated with an ongoing procedure. In an embodiment, each contributor may be able to switch between different relevant views such as real-time video or images, three-dimensional models, preprocedural images, or other relevant multimedia.

The telepresence interface 700 may furthermore include a telepresence content feed 715 for sending and receiving real-time messages between contributors. For example, a telepresence content feed 715 allows users to post messages and/or view messages from other participants. The messages may include text, media content (e.g., images, video, animations, etc.), or links to various media content or other resources (e.g., research articles). The telepresence interface 700 may furthermore enable participants to provide annotations on the target anatomy (presented in the form of an image, video, or model). For example, a participant may pin a comment to a specific location in the depicted anatomy, as may be indicated by an identifier 710.

Additionally, the telepresence interface 700 may display statistics 720 or other analytics that may be relevant to the procedure. The statistics 720 maybe include estimated or modeled values or metrics relating to the anatomy based on various sensed data from the medical equipment 160. The telepresence interface 700 may dynamically update the statistics 720 over time during the procedure.

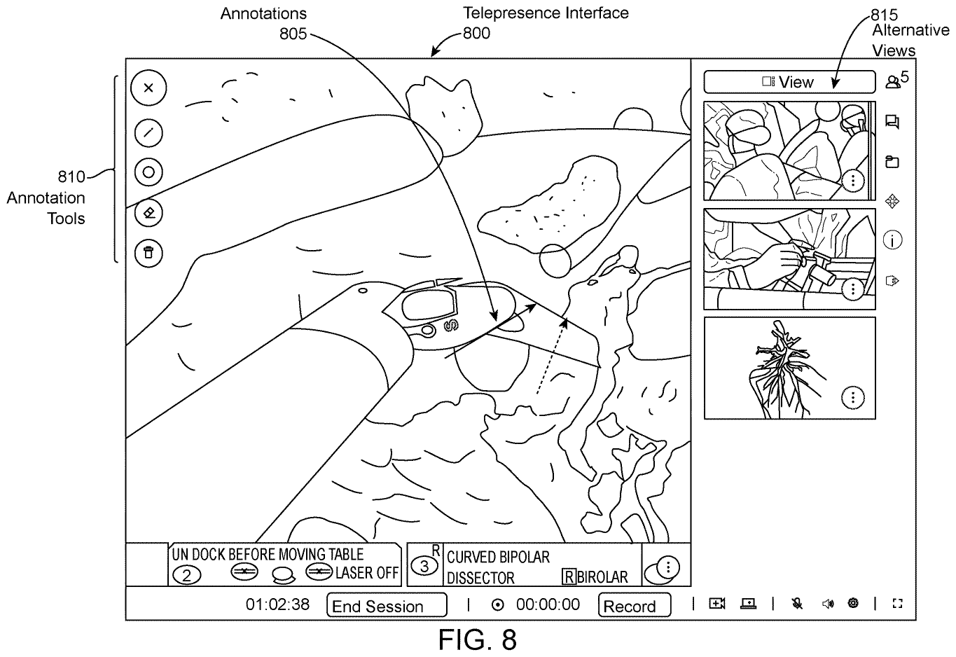

FIG. 8 is another example of a telepresence interface 800 associated with a telepresence session. In this example, the telepresence interface 800 shows a live video of a procedure being performed together with a set of annotation tools 810 that enables a remote contributor to add annotation 805 overlaid on the video. The telepresence interface 800 also includes a set of alternative views 815 the contributor can switch between during the telepresence session. These alternative views 815 may include one or more different camera views (e.g., a view of the medical environment), one or more three-dimensional models (e.g., as shown in FIG. 7), views of preprocedural images, or other multimedia associated with the case. In various embodiments, telepresence interfaces, such as shown in FIG. 7 or 8, are stored for a case and may be subsequently presented to other medical practitioners if a medical practitioner associated with the case authorizes generation of a reference case based on the case, as further described above in conjunction with FIG. 2.

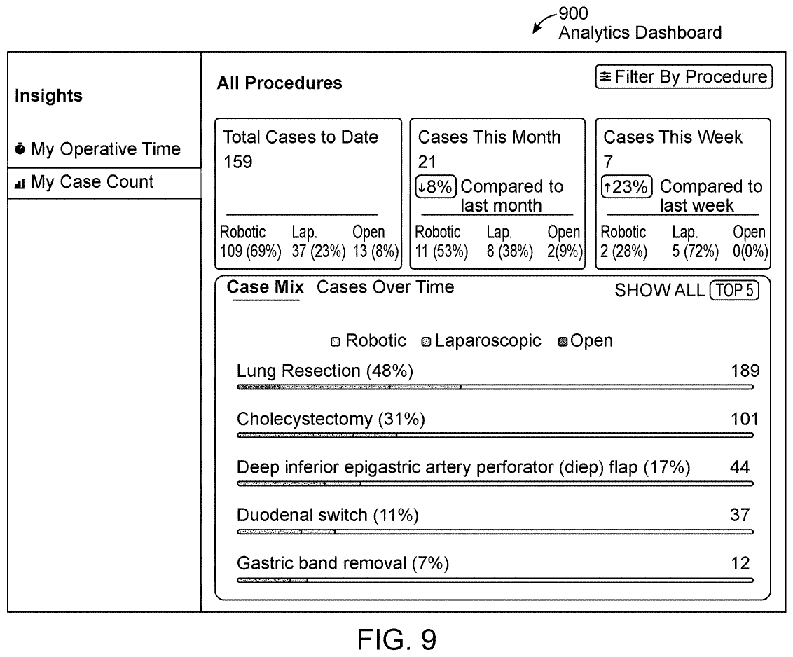

FIG. 9 is an example embodiment of an analytics dashboard 900 for a medical practitioner. In this example, the analytics dashboard 900 displays a summary of cases managed by the medical practitioner includes, for example, a total number of cases, a number of cases in the current month, a number of cases in the current week, and a distribution of types of cases the practitioner has performed.

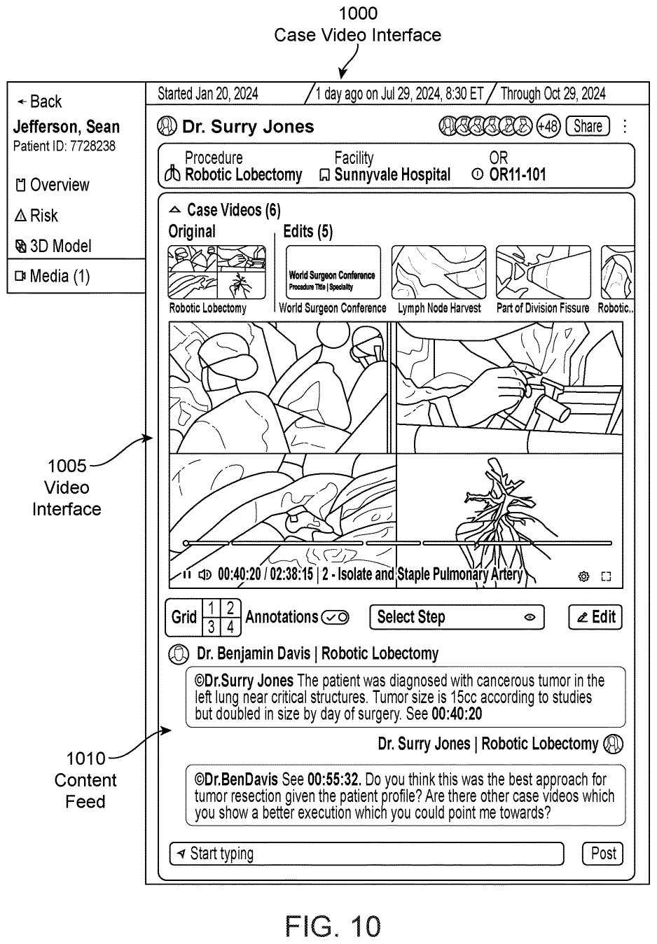

FIG. 10 is an example embodiment of case video interface dashboard 1000 for viewing a case video. Case videos may be captured during a telepresence session or may be similarly captured during a procedure without a live streamed telepresence session. The case video interface 1000 includes a video interface that shows one or more views of a video associated with a medical procedure. The video interface 1000 may include multiple captured views, which may be from cameras in the medical environment, cameras inserted into the anatomy (e.g., endoscopy cameras), or other cameras. Captured views may furthermore include three-dimensional models, preprocedural images, procedure planning documents, or other visual information. The video may be segmented (manually or automatically using video processing and content recognition techniques) to divide the video into segments associated with different steps of the procedure. The video may include annotations provided by a medical practitioner during a telepresence session or in a postprocedural review. A content feed 1010 may be presented in association with a video to enable users 155 to post comments, links, media, or other content in association with the presentation. A reference content item, such as a reference case, may display video and other information (e.g., a content feed 1010) of a medical procedure to a medical practitioner using the video interface 1000 described in conjunction with FIG. 10.

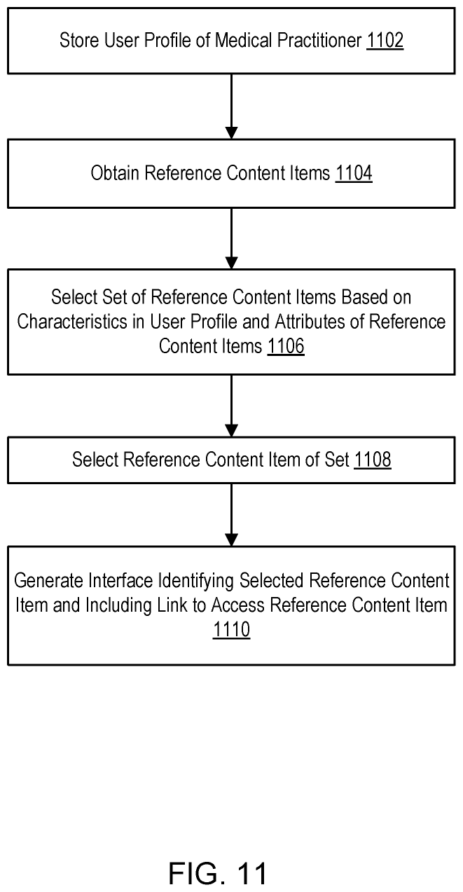

FIG. 11 is an example embodiment of a process for selecting one or more reference content items for presentation to a medical practitioner using a collaborative medical platform 140. The collaborative medical platform 140 stores 1102 a user profile for a medical practitioner including various characteristics of the medical practitioner. For example, the user profile includes an area of specialization of the medical practitioner, types of medical procedures associated with the medical practitioner (e.g., scheduled to be performed by the medical practitioner, previously performed by the medical practitioner), a location where the medical practitioner performs medical procedures, one or more collaborators connected to the medical practitioner through the collaborative medical platform, or other information describing performance of medical procedures by the medical practitioner. Other descriptive information about the medical practitioner, such as performance metrics from previously performed medical procedures may also be stored in the user profile.

The collaborative medical platform 140 also obtains 1104 reference content items. One or more reference content items comprise reference cases generated from medical cases with previously completed medical procedures. A reference case includes data captured by the collaborative medical platform 140 during performance of a previously completed medical procedure. Reference content items may include text data, audio data, video, data, or a combination thereof related to a medical procedure. Reference content items may be locally stored by the collaborative medical platform 140 or may be obtained from one or more third-party servers 170 in various embodiments. Each reference content item has one or more attributes. Example attributes of a reference content item include: a type of medical procedure associated with the reference content item, a location associated with the reference content item, one or more medical practitioners associated with the reference content item, feedback received from other medical practitioners, or other descriptive information about the reference content item.

Based on characteristics of the medical practitioner from the user profile and attributes of reference content items, the collaborative medical platform 140 selects 1106 a set of reference content items for the medical practitioner. As further described above in conjunction with FIG. 2, the collaborative medical platform 140 selects 1106 a set of reference content items having an attribute that at least partially matches a characteristic of the medical practitioner from the user profile. For example, the collaborative medical platform 140 selects 1106 reference content items that each have an attribute including a type of medical procedure that is associated with an area of specialization of the medical practitioner in the user profile.

From the set of reference content items, the collaborative medical platform 140 selects 1108 a reference content item for presentation to the medical practitioner. The collaborative medical platform 140 uses attributes of reference content items and characteristics of the medical practitioner to select 1108 the reference content item of the set. For example, the collaborative medical platform 140 selects 1108 a reference content item associated with a collaborator with the medical practitioner one or more medical cases. As another example, the collaborative medical platform 140 selects 1108 a reference content item associated with a type of medical procedure matching a medical procedure scheduled to be performed by the medical practitioner. In some embodiments, the collaborative medical platform 140 selects a reference content item associated with a type of medical procedure matching a medical procedure scheduled to be performed by an additional medical practitioner connected to the medical practitioner via the collaborative medical platform 140 (e.g., an additional medical practitioner being supervised by the medical practitioner, an additional medical practitioner performing the medical procedure in conjunction with the medical practitioner, etc.). In other embodiments, the collaborative medical platform 140 generates scores or predicted likelihoods of being accessed by the medical practitioner for various reference content items of the set and selects 1108 a reference content item of the set based on the scores or predicted likelihoods of being accessed by the medical practitioner. The collaborative medical platform 140 may account for prior accesses of or interactions with reference content items when selecting 1108 the reference content item to increase a likelihood of the medical practitioner accessing the selected reference content item in various embodiments.

The collaborative medical platform generates 1110 an interface identifying the selected reference content item and including a link to access the selected reference content for presentation to the medical practitioner. For example, the collaborative medical platform 140 generates a practitioner dashboard including a section identifying the selected reference content item. The practitioner dashboard, or other interface, includes a link that retrieves the selected reference content item for presentation when selected by the medical practitioner. In some embodiments, the interface identifies the selected reference content item at a specific time, such as at a time that is a determined time interval before a procedure time when the medical practitioner is scheduled to perform a medical procedure. Different interfaces may be generated 1110 to identify the selected reference content in different embodiments.

The described embodiments incorporate multiple technical improvements that improve the functioning of computer systems, machine learning techniques, data management systems (particularly as related to healthcare data management), computer-based user interfaces, robotic and/or other medical instrumentation systems, and other technologies and technical fields. For example, the described embodiments provide technical improvements in data availability and data privacy by enabling automated processing of sensitive and/or restricted data such as operating room video, patient health records, or other sensitive health data.

The described embodiments furthermore include improvements in machine learning methods in that they combine information from disparate data sources including medical equipment telemetry data, video data, and mobile device data to improve predictive power of machine learning models relative to traditional machine learning techniques. Further still, the described embodiments provide technical improvements in treatment of medical conditions by enabling generation of various notifications, recommendations, or other content tailored to specific medical practitioners that enable them to improve their practice and accordingly results in better patient outcomes.

Furthermore, the described embodiments include technical improvements in the field of robotic-assisted surgery by enabling automated configuration of surgical robots based on the accumulated and aggregated healthcare data associated with patients, medical facilities, and medical practitioners. This results in improved performance of one or more surgical robots, improved human-robot interactions, and improved patient outcomes.

The foregoing description of the embodiments has been presented for the purpose of illustration; it is not intended to be exhaustive or to limit the embodiments to the precise forms disclosed. Persons skilled in the relevant art can appreciate that many modifications and variations are possible in light of the above disclosure.

Some portions of this description describe the embodiments in terms of algorithms and symbolic representations of operations on information. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. Embodiments may also relate to an apparatus for performing the operations herein. This apparatus may be specially constructed for the required purposes, and/or it may include a general-purpose computing device selectively activated or reconfigured by a computer program stored in the computer. Such a computer program may be stored in a tangible non-transitory computer readable storage medium or any type of media suitable for storing electronic instructions and coupled to a computer system bus. Furthermore, any computing systems referred to in the specification may include a single processor or may include architectures employing multiple processor designs for increased computing capability.

Finally, the language used in the specification has been principally selected for readability and instructional purposes, and it may not have been selected to delineate or circumscribe the inventive subject matter. It is therefore intended that the scope is not limited by this detailed description, but rather by any claims that issue on an application based hereon. Accordingly, the disclosure of the embodiments is intended to be illustrative, but not limiting, of the scope of the invention, which is set forth in the following claims.

What is claimed is:

1. A method for identifying one or more reference content items through an online collaborative medical platform, the method comprising:
storing to a database of the online collaborative medical platform, a user profile of a medical practitioner, the user profile storing characteristics of the medical practitioner including types of medical procedures associated with the medical practitioner;
obtaining reference content items at the online collaborative medical platform, each reference content item having one or more attributes each identifying a type of medical procedure associated with a reference content item;
selecting a set of reference content items, each reference content item of the set having at least one attribute matching a type of medical procedure associated with the medical practitioner;
selecting a reference content item of the set based on characteristics of the medical practitioner included in the user profile; and
generating an interface for presentation to the medical practitioner identifying the selected reference content item, the interface including a link for retrieving the selected reference content item when selected by the medical practitioner.

2. The method of claim 1, wherein selecting the set of reference content items comprises:
selecting reference content items having an attribute identifying a type of medical procedure that is associated with an area of specialization included in the user profile of the medical practitioner.

3. The method of claim 1, wherein the user profile of the medical practitioner includes a medical procedure scheduled to be performed by the medical practitioner at a time, and selecting the set of reference content items comprises:
identifying one or more reference content items associated with a type of the medical procedure scheduled to be performed by the medical practitioner.

4. The method of claim 3, wherein generating the interface for presentation to the medical practitioner comprises:
determining a time interval before the time when the medical procedure is scheduled to be performed by the medical practitioner by applying a machine learned model to prior time intervals between times when the user previously accessed reference content items associated with a type of prior medical procedure before procedure times when the medical practitioner performed prior medical procedures having the type; and
generating the interface for presentation to the medical practitioner identifying the selected reference content item during the time interval before the time when the medical procedure is scheduled to be performed by the medical practitioner.

5. The method of claim 1, wherein selecting the set of reference content items comprises:
selecting reference content items having an attribute identifying a type of medical procedure that is associated with an area of specialization included in the user profile of the medical practitioner and having another attribute identifying a location in the user profile where the medical practitioner performs medical procedures.

6. The method of claim 1, wherein selecting the reference content item of the set based on characteristics of the medical practitioner included in the user profile comprises:
selecting a reference content item associated with an additional medical practitioner identified in the user profile as a collaborator with the medical practitioner on a medical case scheduled to be performed by the medical practitioner.

7. The method of claim 1, wherein selecting the set of reference content items comprises:
determining a performance metric for a medical practitioner based on data captured during performance of a medical procedure by the medical practitioner; and
selecting the set of reference content items in response to the performance metric satisfying one or more criteria.

8. The method of claim 1, wherein selecting the reference content item of the set based on characteristics of the medical practitioner included in the user profile comprises:
determining a preferred format of reference content items for the medical practitioner by applying a machine learning model to formats of reference content items the medical practitioner previously accessed; and
selecting a reference content item of the set having the preferred format of reference content items for the medical practitioner.

9. The method of claim 1, wherein selecting the reference content item of the set based on characteristics of the medical practitioner included in the user profile comprises:
generating a predicted likelihood of the medical practitioner accessing each reference content item of the set by applying a machine learning model to each combination of the medical practitioner and a reference content item of the set; and
selecting the reference content based on the predicted likelihoods.

10. The method of claim 1, wherein selecting the reference content item of the set based on characteristics of the medical practitioner included in the user profile comprises:
determining telemetry data captured from a medical robot during a medical procedure performed by the medical practitioner identifies a specific configuration of the medical robot; and
selecting a reference content item of the set associated with the specific configuration of the medical robot.

11. The method of claim 1, wherein selecting the reference content item of the set based on characteristics of the medical practitioner included in the user profile comprises:
determining telemetry data captured from a piece of medical equipment during a medical procedure performed by the medical practitioner identifies a specific pattern or movement of a portion of medical equipment; and
selecting a reference content item of the set associated with movement of the portion of medical equipment.

12. A non-transitory computer-readable storage medium storing instructions for identifying one or more reference content items through an online collaborative medical platform, the instructions when executed by one or more processors causing the one or more processors to perform steps including:
storing to a database of the online collaborative medical platform, a user profile of a medical practitioner, the user profile storing characteristics of the medical practitioner including types of medical procedures associated with the medical practitioner;
obtaining reference content items at the online collaborative medical platform, each reference content item having one or more attributes each identifying a type of medical procedure associated with a reference content item;
selecting a set of reference content items, each reference content item of the set having at least one attribute matching a type of medical procedure associated with the medical practitioner;
selecting a reference content item of the set based on characteristics of the medical practitioner included in the user profile; and
generating an interface for presentation to the medical practitioner identifying the selected reference content item, the interface including a link for retrieving the selected reference content item when selected by the medical practitioner.

13. The non-transitory computer-readable storage medium of claim 12, wherein selecting the set of reference content items comprises:
selecting reference content items having an attribute identifying a type of medical procedure that is associated with an area of specialization included in the user profile of the medical practitioner.

14. The non-transitory computer-readable storage medium of claim 12, wherein the user profile of the medical practitioner includes a medical procedure scheduled to be performed by the medical practitioner at a time, and selecting the set of reference content items comprises:
identifying one or more reference content items associated with a type of the medical procedure scheduled to be performed by the medical practitioner.

15. The non-transitory computer-readable storage medium of claim 14, wherein generating the interface for presentation to the medical practitioner comprises:
determining a time interval before the time when the medical procedure is scheduled to be performed by the medical practitioner by applying a machine learned model to prior time intervals between times when the user previously accessed reference content items associated with a type of prior medical procedure before procedure times when the medical practitioner performed prior medical procedures having the type; and
generating the interface for presentation to the medical practitioner identifying the selected reference content item during the time interval before the time when the medical procedure is scheduled to be performed by the medical practitioner.

16. The non-transitory computer-readable storage medium of claim 11, wherein selecting the set of reference content items comprises:
selecting reference content items having an attribute identifying a type of medical procedure that is associated with an area of specialization included in the user profile of the medical practitioner and having another attribute identifying a location in the user profile where the medical practitioner performs medical procedures.

17. The non-transitory computer-readable storage medium of claim 12, wherein selecting the reference content item of the set based on characteristics of the medical practitioner included in the user profile comprises:
selecting a reference content item associated with an additional medical practitioner identified in the user profile as a collaborator with the medical practitioner on a medical case scheduled to be performed by the medical practitioner.

18. The non-transitory computer-readable storage medium of claim 12, wherein selecting the set of reference content items comprises:
determining a performance metric for a medical practitioner based on data captured during performance of a medical procedure by the medical practitioner; and
selecting the set of reference content items in response to the performance metric satisfying one or more criteria.

19. The non-transitory computer-readable storage medium of claim 12, wherein selecting the reference content item of the set based on characteristics of the medical practitioner included in the user profile comprises:
determining a preferred format of reference content items for the medical practitioner by applying a machine learning model to formats of reference content items the medical practitioner previously accessed; and
selecting a reference content item of the set having the preferred format of reference content items for the medical practitioner.

20. The non-transitory computer-readable storage medium of claim 12, wherein selecting the reference content item of the set based on characteristics of the medical practitioner included in the user profile comprises:
generating a predicted likelihood of the medical practitioner accessing each reference content item of the set by applying a machine learning model to each combination of the medical practitioner and a reference content item of the set; and
selecting the reference content based on the predicted likelihoods.

21. The non-transitory computer-readable storage medium of claim 12, wherein selecting the reference content item of the set based on characteristics of the medical practitioner included in the user profile comprises:
determining telemetry data captured from a medical robot during a medical procedure performed by the medical practitioner identifies a specific configuration of the medical robot; and
selecting a reference content item of the set associated with the specific configuration of the medical robot.

22. The non-transitory computer-readable storage medium of claim 12, wherein selecting the reference content item of the set based on characteristics of the medical practitioner included in the user profile comprises:
determining telemetry data captured from a piece of medical equipment during a medical procedure performed by the medical practitioner identifies a specific pattern or movement of a portion of medical equipment; and
selecting a reference content item of the set associated with movement of the portion of medical equipment.
